(19) United States
(12) Patent Application Publication
(10) Pub. No.: US 2010/0069384 A1
(43) Pub. Date: Mar. 18, 2010

(54) 5-ALKYLOXY-INDOLIN-2-ONE DERIVATIVES, PREPARATION THEREOF AND APPLICATION THEREOF IN THERAPY

(75) Inventors: Loic FOULON, Portet Sur Garonne (FR); Pierrick ROCHARD, Roques-Sur-Garonne (FR); Claudine SERRADEIL-LE GAL, GIF SUR YVETTE (FR); Gerard VALETTE, Lacroix-Falgarde (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/483,782

(22) Filed: Jun. 12, 2009

Related U.S. Application Data
(63) Continuation of application No. PCT/FR2007/002028, filed on Dec. 10, 2007.

(30) Foreign Application Priority Data
Dec. 12, 2006 (FR) 0610803

(51) Int. Cl. A61K 31/5377; A61K 31/4439; A61K 31/404; C07D 413/10; C07D 401/10; C07D 209/34; A61P 9/04; A61P 25/22; A61P 25/24; A61P 3/10

(52) U.S. Cl. 514/235.2; 514/339; 514/418; 544/144; 546/277.7; 548/486

(57) ABSTRACT

The present invention relates to derivatives of 5-alkyloxy-indolin-2-one, their method of production and their therapeutic applications. These novel derivatives have affinity and selectivity for the V₂ receptors of vasopressin (“V₂ receptors”) and can therefore constitute active principles of pharmaceutical compositions.

The invention relates to derivatives of 5-alkyloxy-indolin-2-one, their method of production and their therapeutic applications. These novel derivatives have affinity and selectivity for the V₂ receptors of vasopressin (“V₂ receptors”) and can therefore constitute active principles of pharmaceutical compositions.

Vasopressin (V) is a hormone that is known for its antidiuretic effect and its effect in the regulation of arterial pressure. It stimulates several types of receptors: V₁ (V₁ₐ, V_{1b} or V₃), V₂.

Oxytocin (OXT) has a peptide structure similar to that of vasopressin.

The V₁ (V₁ₐ, V_{1b}), V₂ and OXT receptors are localised in common tissues and organs (Jard S. et al., “Vasopressin and oxytocin receptors: an overview in progress” in Endocrinology, Himura H. and Shizume K ed., or in: Pharmacol. Rev., 1991 43 (1), 73-108).

Several documents describe a series of non-peptide compounds possessing affinity for the vasopressin receptors and/or oxytocin receptors, such as WO2006/100080, WO2006/072458, WO2005/030755, WO2006/005609, EP 0 636 608, WO95/18105, WO03/008407, WO93/15051 or WO97/15556.

The aim of the present invention is to find novel compounds having potent selectivity for the V₂ vasopressin receptors, while displaying good pharmacological properties. The compounds according to the invention notably display good metabolic stability, making them particularly suitable for use as a medicinal product.

For this purpose, the inventors have developed a new family of compounds having affinity and selectivity for the V₂ vasopressin receptors. These novel compounds are potent antagonists of binding of the V₂ vasopressin receptors. Moreover, the compounds of formula (I) according to the invention display good metabolic stability, notably on human hepatic microsomes, thus confirming the advantages of these compounds for use as medicinal products.

The invention relates to the compounds of general formula (I) hereunder:

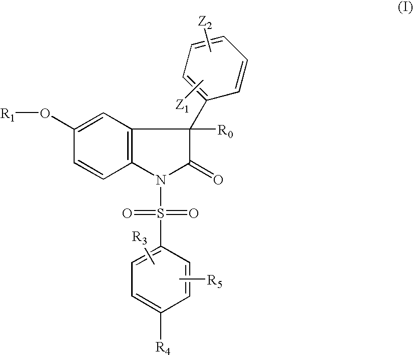

In which

R₀ represents a (C₁-C₄)alkyl group, a mono or polyfluoro-(C₁-C₄)alkyl group, a (CH₂)ₙ-cyclopropyl group,
or alternatively R₀ represents a (C₂-C₄)alkenyl or (C₂-C₄)alkynyl group;

R₁ represents a hydrogen atom, a (C₁-C₅)alkyl group, a mono or polyfluoro-(C₁-C₅)alkyl group, a hydroxy-(C₁-C₅)alkyl group, a —(CH₂)ₘ—(C₃-C₅)cycloalkyl group;

Z₁ represents a hydrogen atom or halogen atom, a (C₁-C₄)alkyl group, a mono or polyfluoro-(C₁-C₄)alkyl group, a (C₁-C₄)alkoxy group, a mono or polyfluoro-(C₁-C₄)alkoxy group, a —(CH₂)ₚ-cyclopropyl group, said cyclopropyl group being unsubstituted or substituted with one or more fluorine atoms;

Z₂ represents a halogen atom or a group T₁W, in which T₁ represents a group —(CH₂)ₙ— and W represents a hydrogen atom, a (C₁-C₄)alkyl group, mono or polyfluoro-(C₁-C₄)alkyl group or a cyclopropyl group unsubstituted or substituted with one or more fluorine atoms,
or alternatively W represents a group —C(O)NR₆R₇ in which R₆ and R₇ represent, independently of one another, a hydrogen atom, a (C₁-C₆)alkyl group, a mono or polyfluoro-(C₁-C₆)alkyl group, a —(CH₂)ₘ—(C₃-C₆)cycloalkyl group, said cycloalkyl group being unsubstituted or substituted with one or more fluorine atoms, a hydroxyl or a group NRR′,
or alternatively R₆ and R₇ represent, independently of one another, a —(CH₂)ₚ-pyrrolidinyl, —(CH₂)ₚ-piperidyl, —(CH₂)ₚ-pyridyl group, said pyrrolidinyl, piperidyl and pyridyl groups being unsubstituted or substituted with one or more halogen atoms, a (C₁-C₄)alkyl group, a mono or polyfluoro(C₁-C₄)alkyl group, a benzyl or by a group —OR,
or alternatively R₆ and R₇ represent, independently of one another, a group —(CH₂)_{q}—NRₐR_{b},
or alternatively R₆ and R₇ represent, independently of one another, a group —(CH₂)ₛ—C(O)NRₐR_{b},
or alternatively R₆ and R₇ represent, independently of one another, a group —(CH₂)_{q}—OR,
or alternatively R₆ and R₇ form, together with the nitrogen atom to which they are attached, a monocyclic heterocycle unsubstituted or substituted with one or more fluorine atoms, one or more (C₁-C₄)alkyl, mono or polyfluoro-(C₁-C₄)alkyl, —NR′R, or —OR groups,
or alternatively R₆ and R₇ form, together with the nitrogen atom to which they are attached, a bicyclic heterocycle unsubstituted or substituted with one or more fluorine atoms, (C₁-C₄)alkyl, mono or polyfluoro-(C₁-C₄)alkyl, —OR or —NR′R groups;
   or alternatively W represents a group —NR₈C(O)R₉ in which:
R₈ represents a hydrogen atom, a (C₁-C₄)alkyl group, a mono or polyfluoro-(C₁-C₄)alkyl group,
R₉ represents a hydrogen atom, a (C₁-C₄)alkyl group, a mono or polyfluoro-(C₁-C₄)alkyl group, —(CH₂)ᵣ—NR_{d}Rₑ, —(CH₂)ₘ-pyrrolidinyl, —(CH₂)ₘ-piperidyl, —(CH₂)ₘ-pyridyl group, said pyrrolidinyl, piperidyl, or pyridyl groups being unsubstituted or substituted with one or more (C₁-C₄)alkyl, halogen, mono or polyfluoro-(C₁-C₄)alkyl, benzyl groups;
   or alternatively W represents a group —NR₁₀R₁₁ in which R₁₀ and R₁₁ represent, independently of one another, a hydrogen atom, a hydroxyl group, a (C₁-C₆)alkyl or a mono or polyfluoro-(C₁-C₆)alkyl group,
or alternatively R₁₀ and R₁₁ form, together with the nitrogen atom to which they are attached, a monocyclic heterocycle unsubstituted or substituted with one or more (C₁-C₄)alkyl groups, a mono or polyfluoro-(C₁-C₄)alkyl group or oxo;
   or alternatively W represents a group —OR₁₂ in which R₁₂ represents a hydrogen atom, a (C₁-C₄)alkyl group, a mono or polyfluoro-(C₁-C₄)alkyl group, a benzyl or a group —(CH₂)_{q}—NR′R;
   or alternatively W represents a group —C(O)OR₁₉.

R₄ represents a (C₁-C₄)alkyl group, a mono or polyfluoro-(C₁-C₄)alkyl group, an —OR, (C₂-C₄) alkenyl, nitro, COOR_{c}, a benzyloxy group,
or alternatively R₄ represents a group —C(O)NR₁₃R₁₄ in which R₁₃ and R₁₄ represent, independently of one another, a hydrogen atom, a (C₁-C₆)alkyl group, said alkyl group being unsubstituted or substituted with one or more fluorine atoms, a hydroxyl, an NRR′ group, a (C₁-C₆)alkyloxycarbonylamino group, a (C₁-C₆)alkyloxycarbonyl group, or in which R₁₃ and R₁₄ represent, independently of one another, a —(CH₂)ₙ—(C₃-C₆)cycloalkyl group, said cycloalkyl group being unsubstituted or substituted with one or more fluorine atoms,
or alternatively R₄ represents a group —NR₁₅R₁₆ in which R₁₅ and R₁₆ represent, independently of one another, a hydrogen atom, a hydroxyl group, a (C₁-C₄)alkyl group, a mono or polyfluoro-(C₁-C₄)alkyl group, a —(CH₂)ₙ—(C₃-C₆)cycloalkyl group unsubstituted or substituted with one or more fluorine atoms,
   or alternatively R₁₅ and R₁₆ form, together with the nitrogen atom to which they are attached, a monocyclic heterocycle,
or alternatively R₄ represents a group —NR₁₇C(O)R₁₈ in which:
   R₁₇ represents a hydrogen atom, a (C₁-C₄)alkyl group, a mono or polyfluoro-(C₁-C₄)alkyl group,
   R₁₈ represents a (C₁-C₆)alkyl group, a mono or polyfluoro-(C₁-C₆)alkyl group,
      —(CH₂)ₙ—(C₃-C₆)cycloalkyl, a group —NR_{d}Rₑ, a phenyl group, said phenyl group itself being unsubstituted or substituted with one or more (C₁-C₄)alkyl groups, a mono or polyfluoro-(C₁-C₄)alkyl group;
R₁₉ represents a hydrogen atom, a (C₁-C₆)alkyl group, a mono or polyfluoro-(C₁-C₆)alkyl group, a group —(CH₂)_{q}—NRₐR_{b}, a group —(CH₂)_{q}—OR, a —(CH₂)ₚ-pyrrolidinyl group or a —(CH₂)ₚ-piperidyl group, said pyrrolidinyl and piperidyl groups being unsubstituted or substituted with one or more fluorine atoms, a (C₁-C₄)alkyl group, a mono or polyfluoro-(C₁-C₄)alkyl group.

R₃ and R₅ represent, independently of one another, a hydrogen atom, a halogen atom, a (C₁-C₄)alkyl group, a mono or polyfluoro-(C₁-C₄)alkyl group, a (C₁-C₄)alkoxy or mono or polyfluoro-(C₁-C₄)alkoxy group;

Rₐ and R_{b} represent, independently of one another:
a hydrogen atom, a (C₁-C₄)alkyl group, a mono or polyfluoro-(C₁-C₄)alkyl group, a —(CH₂)ₙ-cyclopropyl group, said cyclopropyl being unsubstituted or substituted with one or more fluorine atoms,
or alternatively Rₐ, and R_{b} form, together with the nitrogen atom to which they are attached, a monocyclic heterocyclic group, said monocyclic heterocyclic group being unsubstituted or substituted with one or more hydroxyl, (C₁-C₄)alkyl, mono or polyfluoro-(C₁-C₄)alkyl, (C₁-C₄)alkoxy, mono or polyfluoro(C₁-C₄)alkoxy groups, or by a group —NRR′;

R′ and R represent, independently of one another, a hydrogen atom, a (C₁-C₄)alkyl group or mono or polyfluoro-(C₁-C₄)alkyl group;

R_{c} represents a hydrogen atom, a (C₁-C₄)alkyl group, mono or polyfluoro-(C₁-C₄)alkyl group or a benzyl group;

R_{d} and Rₑ represent, independently of one another, a hydrogen, atom, a halogen atom, a (C₁-C₆)alkyl group or mono or polyhalogen-(C₁-C₆)alkyl group,
or alternatively R_{d} and Rₑ form, together with the nitrogen atom to which they are attached, a monocyclic heterocyclic group, unsubstituted or substituted with one or more fluorine atoms, one or more (C₁-C₄)alkyl or mono or polyfluoro-(C₁-C₄)alkyl groups or a group —OR;

m can represent the value 0, 1 or 2;

n can represent the value 0 or 1;

p can represent the value 0, 1, 2 or 3;

q can represent the value 2, 3, 4 or 5;

r can represent the value 0, 1, 2, 3 or 4;

s can represent the value 1, 2 or 3.

The compounds of general formula (I) can occur in the form of tautomers. Thus, the invention relates to the compounds of the invention in all their tautomeric forms.

The compounds of general formula (I) can have one or more asymmetric carbon atoms. They can therefore be in the form of enantiomers or of diastereoisomers. These enantiomers, diastereoisomers, and mixtures thereof, including racemic mixtures, form part of the invention.

The compounds of general formula (I) can contain double bonds or one or more saturated rings. They can therefore be in the form of cis/trans isomers. These isomers, and mixtures thereof, form part of the invention.

The compounds of general formula (I) can occur as bases or acids and salts of addition. Said salts of addition form part of the invention.

These salts are advantageously prepared with pharmaceutically acceptable acids or bases, but the salts of other acids or bases that can be used, for example, for purifying or isolating the compounds of general formula (I) also form part of the invention.

The compounds of general formula (I) can be in the form of hydrates or of solvates, namely in the form of associations or combinations with one or more molecules of water or with a solvent. Said hydrates and solvates also form part of the invention.

According to the present invention, the N-oxides of the compounds bearing an amine also form part of the invention.

The compounds of formula (I) according to the present invention also include those in which one or more atoms of hydrogen, carbon or halogen, notably of chlorine or of fluorine have been replaced by their radioactive isotopes, for example tritium to replace hydrogen or carbon 14 to replace carbon 12. Said labelled compounds are useful in research, in studies of metabolism or of pharmacokinetics, and in biochemical assays as ligands of receptors.

The prodrugs of the compounds of formula (I) according to the present invention also form part of the invention. By prodrugs, we mean compounds that are metabolised in vivo to compounds of the invention. Examples of prodrugs are described notably in John B. Taylor (Vol. Ed): Comprehensive Medicinal Chemistry, Pergamon Press, 1990, Vol. 5 p. 122-132 and in C. Wermuth (Ed.): The Practice of Medicinal Chemistry, Elsevier Academic Press, 2003, p. 561-585. As examples of prodrugs, we may mention compounds in which a —COO(C₁-C₆)alkyl ester group, a —OC(O)R group or an amino group in which one or two hydrogens are replaced by (C₁-C₆)alkyl groups are the prodrugs of compound (I) according to the invention, containing respectively a —COOH carboxylic acid group, an —OH alcohol group or a primary amine group —NH₂. Moreover, the compounds of the invention according to (I) can themselves behave in vivo as prodrugs of other compounds of the invention according to (I). As an example, we may mention an aminocarbonyl group —C(O)NR₆R₇, R₆ and R₇ being defined as previously and can lead to a compound (I) according to the invention having a —COOH group.

Within the scope of the invention:
C₁-C_{z} where t and z can take the values from 1 to 6, denotes a carbon chain that can have from t to z carbon atoms, for example C₁—6 represents a carbon chain that can have from 1 to 6 carbon atoms or alternatively

C₁-C₃ represents a carbon chain that can have from 1 to 3 carbon atoms;
or alternatively Cₓ-C_{y} where x and y can take the values from 3 to 6, denotes a saturated carbon ring that can have from x to y carbon atoms, for example C₃₋₆ represents a saturated carbon ring that can have from 3 to 6 carbon atoms;
alkyl denotes a linear or branched, saturated aliphatic group; for example, a group C₁-C₆ alkyl represents a linear or branched carbon chain of 1 to 6 carbon atoms, notably a methyl, ethyl, propyl, isopropyl, butyl, isobutyl, secbutyl, tertbutyl, pentyl;
an alkenyl group denotes a linear or branched, mono- or poly-unsaturated aliphatic group, comprising for example one or two ethylenic unsaturations. For example, a group C₂-C₆ alkenyl can represent an ethenyl, propenyl, and so on;
an alkynyl group denotes a linear or branched, mono- or poly-unsaturated aliphatic group, comprising for example one or two acetylenic unsaturations. For example, a group C₂-C₆ alkynyl can represent an ethynyl, propynyl, and so on;
alkoxy denotes an alkyloxy group with a linear or branched saturated aliphatic chain;
halogen atom: an atom of fluorine, chlorine, bromine or iodine;
a polyfluoroalkyl group: an alkyl group as defined previously, in which one or more hydrogen atoms are replaced by a fluorine atom respectively. As an example we may mention the trifluoromethyl group (—CF₃) in which 3 hydrogen atoms have been replaced by 3 fluorine atoms;
a polyfluoroalkoxy group: an alkoxy group as defined previously, in which one or more hydrogen atoms are replaced by a fluorine atom respectively. As an example we may mention the trifluoromethoxy group (—OCF₃) in which 3 hydrogen atoms have been replaced by 3 fluorine atoms;
a monocyclic heterocyclic group: a saturated monocyclic group with 5 to 7 ring members having one or more heteroatoms such as nitrogen, oxygen or sulphur atoms. As examples, we may mention the pyrrolidinyl, dihydropyrrolidinyl, piperidyl, tetrahydropyranyl, morpholinyl, piperazinyl, tetrahydrofuranyl, thiomorpholine, azepinyl, homomorpholinyl, homopiperazinyl groups;
a bicyclic heterocycle: a bicyclic structure comprising one or more heteroatoms such as nitrogen, oxygen or sulphur atoms, said structure being composed of 2 saturated or partially unsaturated cyclic groups, fused (i.e. said groups have a bond in common) or bridged (i.e. at least 2 atoms of the bicyclic structure are joined by a single bond or a carbon chain that can have from 1 to 4 carbon atoms), and can have from 6 to 18 ring members.

As an example of a saturated fused bicyclic heterocycle, we may mention the octahydro-pyrrolo[3,4-b]pyrrole group:

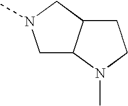

As an example of a saturated bridged bicyclic heterocycle, we may mention the 2,5-diazabicyclo[2.2.1]heptane group:

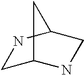

The present invention also relates to methods of preparation of the compounds of general formula (I).

Thus, the compounds of the invention can be prepared by the methods shown in the schemes given below, the operating conditions of which are conventional for a person skilled in the art.

Protecting group PG means a group that is able to prevent the reactivity of a function or position, during a chemical reaction that might affect it, and that restores the molecule after cleavage according to methods known by a person skilled in the art. Temporary protecting groups of amines or alcohols means the protecting groups such as those described in Protective Groups in Organic Synthesis, Greene T. W. and Wuts P. G. M., Ed. Wiley Intersciences 1999 and in Protecting Groups, Kocienski P. J., 1994, Georg Thieme Verlag.

We may mention for example temporary protecting groups of the amines: benzyls, carbamates (such as tert-butyloxycarbonyl groups that can be cleaved in an acid environment, benzyloxycarbonyl groups that can be cleaved by hydrogenolysis), temporary protecting groups of carboxylic acids: alkyl esters (such as methyl or ethyl, tert-butyl hydrolysable in a basic or acid medium) and hydrogenolysable benzyl esters, temporary protecting groups of alcohols or of phenols such as the tetrahydropyranyl, methyloxymethyl or methylethoxymethyl, tert-butyl and benzyl ethers, temporary protecting groups of carbonylated derivatives such as the linear or cyclic acetals, for example 1,3-dioxan-2-yl or 1,3-dioxolan-2-yl; and reference may be made to the well-known general methods described in Protective Groups, cited above.

A person skilled in the art will be able to choose the appropriate protecting groups. The compounds of formula (I) can include groups that are precursors of other functions that are generated subsequently in one or more other stages.

Leaving group means, hereinafter, a group that can easily be cleaved by heterolytic bond rupture; we may mention for example the halogens (I, Br, CI, F) or an activated hydroxyl group such as a methanesulphonate, benzenesulphonate, p-toluenesulphonate, triflate, acetate, etc. Examples of leaving groups as well as references for their preparation are given in “March's Advanced Organic Chemistry”, J. March, 5^{th} Edition, Wiley Interscience, p. 449.

Precursor group means, hereinafter, any group that can be transformed in one or more stages to another group by the chemical reactions known by a person skilled in the art. Among the compounds according to the invention, we may mention a first group of compounds of general formula (I), in which R₀ represents a (C₁-C₃)alkyl group, in particular methyl, the other groups being as defined for the compound of general formula (I).

Among the compounds according to the invention, we may mention a second group of compounds of general formula (I), in which R₀ represents a (C₁-C₃)alkyl group, in particular methyl, and R₁ represents a (C₁-C₅)alkyl group, the other groups being as defined for the compound of general formula (I).

Among the compounds according to the invention, we may mention a third group of compounds of general formula (I), in which R₀ represents a methyl group and R₁ represents a (C₁-C₂)alkyl group, more particularly ethyl, the other groups being as defined for the compound of general formula (I).

Among the compounds according to the invention, we may mention a fourth group of compounds of general formula (I), in which Z₁ represents a halogen atom, more particularly a fluorine atom or a chlorine atom, the other groups being as defined for the compound of general formula (I).

Among the compounds according to the invention, we may mention a fifth group of compounds of general formula (I), in which Z₂ represents a group T₁W, in which T₁ represents a group —(CH₂)ₙ— with n equal to 0 and W represents:
a group —C(O)OR₁₉,
a group —C(O)NR₆R₇,
   in which R₁₉, R₆ and R₇ and the other groups are as defined for the compound of general formula (I).

Among the compounds according to the invention, we may mention a sixth group of compounds of general formula (I), in which Z₁ is in position −2.

Among the compounds according to the invention, we may mention a seventh group of compounds of general formula (I), in which Z₁ is in position −2 and Z₂ is in position −5.

Among the compounds according to the invention, we may mention an eighth group of compounds of general formula (I), in which R₄ represents a group —C(O)NR₁₃R₁₄, in which R₁₃, R₁₄ and the other groups are as defined for the compound of general formula (I).

Among the compounds according to the invention, we may mention a ninth group of compounds of general formula (I), in which Z₂ represents a group T₁W, in which T₁ represents a group —(CH₂)ₙ— with n equal to 0 and W represents a group —OR₁₂ in which R₁₂ represents a hydrogen atom.

Among the compounds according to the invention, we may mention a tenth group of compounds of general formula (I), in which Z₁ is in position −2 and Z₂ represents a halogen.

Among the compounds in accordance with the invention, we may mention the following compounds:
N-tert-butyl-4-[3-(2-chlorophenyl)-5-ethoxy-3-methyl-2-oxo-2,3-dihydroindole-1-sulphonyl]benzamide (Example 2)
N-tert-butyl-4-[3-(2-fluorophenyl)-5-ethoxy-3-methyl-2-oxo-2,3-dihydroindole-1-sulphonyl]benzamide (Example 6)
methyl 3-[1-(4-tert-butylcarbamoylbenzenesulphonyl)-5-ethoxy-3-methyl-2-oxo-2,3-dihydro-1H-indol-3-yl]-4-chlorobenzoate (Example 9)
N-tert-butyl-4-[3-(2-chlorophenyl)-5-ethoxy-3-methyl-2-oxo-2,3-dihydroindole-1-sulphonyl]benzamide (Example 12)
N-cyclopentyl-4-[3-(2-chlorophenyl)-5-ethoxy-3-methyl-2-oxo-2,3-dihydroindole-1-sulphonyl]benzamide (Example 13)
N-tert-butyl-3-methoxy-4-[3-(2-chlorophenyl)-5-ethoxy-3-methyl-2-oxo-2,3-di-hydroindole-1-sulphonyl]benzamide (Example 15)
N-tert-butyl-3-methoxy-4-[3-(2-fluorophenyl)-5-ethoxy-3-methyl-2-oxo-2,3-dihydro-indole-1-sulphonyl]benzamide (Example 16)
3-[1-(4-tert-butylcarbamoyl-2-methoxybenzenesulphonyl)-5-ethoxy-3-methyl-2-oxo-2,3-dihydro-1H-indol-3-yl]-4-chlorobenzoic acid (Example 19)
4-chloro-3-[1-(4-tert-butylcarbamoyl-2-methoxybenzenesulphonyl)-5-ethoxy-3-methyl-2-oxo-2,3-dihydro-1H-indol-3-yl]-N-ethylbenzamide (Example 21)
4-chloro-3-[1-(4-tert-butylcarbamoyl-2-methoxybenzenesulphonyl)-5-ethoxy-3-methyl-2-oxo-2,3-dihydro-1H-indol-3-yl]-N-(2-pyridyl)benzamide (Example 22)
4-chloro-3-[1-(4-tert-butylcarbamoyl-2-methoxybenzenesulphonyl)-5-ethoxy-3-methyl-2-oxo-2,3-dihydro-1H-indol-3-yl]-N-(3-dimethylaminopropyl)benzamide (Example 25)
4-chloro-3-[1-(4-tert-butylcarbamoyl-2-methoxybenzenesulphonyl)-5-ethoxy-3-methyl-2-oxo-2,3-dihydro-1H-indol-3-yl]-N-(1-methylpiperidin-4-yl)benzamide (Example 28)
4-chloro-3-[1-(4-tert-butylcarbamoyl-2-methoxybenzenesulphonyl)-5-ethoxy-3-methyl-2-oxo-2,3-dihydro-1H-indol-3-yl]-N-(2-pyrrolidin-1-ylethyl)benzamide (Example 29)
4-chloro-3-[1-(4-tert-butylcarbamoyl-2-methoxybenzenesulphonyl)-5-ethoxy-3-methyl-2-oxo-2,3-dihydro-1H-indol-3-yl]-N-(2-diethylaminoethyl)benzamide (Example 33)
4-chloro-3-[1-(4-tert-butylcarbamoyl-2-methoxybenzenesulphonyl)-5-ethoxy-3-methyl-2-oxo-2,3-dihydro-1H-indol-3-yl]-N-(2-morpholin-4-ylethyl)benzamide (Example 36)
4-chloro-3-[1-(4-tert-butylcarbamoyl-2-methoxybenzenesulphonyl)-5-ethoxy-3-methyl-2-oxo-2,3-dihydro-1H-indol-3-yl]-N-[2-(4-methylpiperazin-1-yl)ethyl]benzamide (Example 37)
4-chloro-3-[1-(4-tert-butylcarbamoyl-2-methoxybenzenesulphonyl)-5-ethoxy-3-methyl-2-oxo-2,3-dihydro-1H-indol-3-yl]-N-[3-(4-methylpiperazin-1-yl)propyl]benzamide (Example 40)
4-chloro-3-[1-(4-tert-butylcarbamoyl-2-methoxybenzenesulphonyl)-5-ethoxy-3-methyl-2-oxo-2,3-dihydro-1H-indol-3-yl]-N—(R)-pyrrolidin-3-ylbenzamide (Example 43)
4-chloro-3-[1-(4-tert-butylcarbamoyl-2-methoxybenzenesulphonyl)-5-ethoxy-3-methyl-2-oxo-2,3-dihydro-1H-indol-3-yl]-N-piperidin-4-ylbenzamide (Example 44)
4-chloro-3-[1-(4-tert-butylcarbamoyl-2-methoxybenzenesulphonyl)-5-ethoxy-3-methyl-2-oxo-2,3-dihydro-1H-indol-3-yl]-N-2-aminoethylbenzamide (Example 47)
3-[1-(4-tert-butylcarbamoylbenzenesulphonyl)-5-ethoxy-3-methyl-2-oxo-2,3-di-hydro-1H-indol-3-yl]-4-chlorobenzoic acid (Example 48)
4-chloro-3-[1-(4-tert-butylcarbamoylbenzenesulphonyl)-5-ethoxy-3-methyl-2-oxo-2,3-dihydro-1H-indol-3-yl]-N-(1-methylpiperidin-4-yl)benzamide (Example 51)
4-chloro-3-[1-(4-tert-butylcarbamoylbenzenesulphonyl)-5-ethoxy-3-methyl-2-oxo-2,3-dihydro-1H-indol-3-yl]-N-(2-diethylaminoethyl)benzamide (Example 53)
N-tert-butyl-4-{3-[2-chloro-5-(3-dimethylaminopropionylamino)phenyl]-5-ethoxy-3-methyl-2-oxo-2,3-dihydroindole-1-sulphonyl}-3-methoxybenzamide (Example 58)
1-methylpiperidine-4-carboxylic acid {3-[1-(4-tert-butylcarbamoyl-2-methoxy-benzenesulphonyl)-5-ethoxy-3-methyl-2-oxo-2,3-dihydro-1H-indol-3-yl]-4-chlorophenyl}-amide (Example 62)
N-(2-fluoro-1,1-dimethylethyl)-4-[3-(2-chlorophenyl)-5-ethoxy-3-methyl-2-oxo-2,3-dihydroindole-1-sulphonyl]benzamide (Example 66)
4-[3-(2-chlorophenyl)-5-ethoxy-3-methyl-2-oxo-2,3-dihydroindole-1-sulphonyl]-N-(2-hydroxy-1,1-dimethylethyl)benzamide (Example 72)
4-chloro-3-[1-(4-tert-butylcarbamoylbenzenesulphonyl)-5-ethoxy-3-methyl-2-oxo-2,3-dihydro-1H-indol-3-yl]-N[2-(4-hydroxypiperidin-1-yl)ethyl]benzamide (Example 79)
4-chloro-3-[1-(4-tert-butylcarbamoylbenzenesulphonyl)-5-ethoxy-3-methyl-2-oxo-2,3-dihydro-1H-indol-3-yl]-N-2,2,2-trifluoroethylbenzamide (Example 80)
4-chloro-3-[1-(4-tert-butylcarbamoylbenzenesulphonyl)-5-ethoxy-3-methyl-2-oxo-2,3-dihydro-1H-indol-3-yl]-N-hydroxyethylbenzamide (Example 83)
4-chloro-3-[1-(4-tert-butylcarbamoylbenzenesulphonyl)-5-ethoxy-3-methyl-2-oxo-2,3-dihydro-1H-indol-3-yl]-N-cyclopentylbenzamide (Example 87)
4-chloro-3-[1-(4-tert-butylcarbamoylbenzenesulphonyl)-5-ethoxy-3-methyl-2-oxo-2,3-dihydro-1H-indol-3-yl]-N-[trans-(4-hydroxycyclohexyl)]benzamide (Example 88)
4-chloro-3-[1-(4-tert-butylcarbamoylbenzenesulphonyl)-5-ethoxy-3-methyl-2-oxo-2,3-dihydro-1H-indol-3-yl]-N-[trans-(4-dimethylaminocyclohexyl)]benzamide (Example 90)
N-tert-butyl-4-[3-methyl-3-phenyl-5-ethoxy-2-oxo-2,3-dihydroindole-1-sulphonyl]-benzamide (Example 98)
N-tert-butyl-3-methoxy-4-[3-methyl-3-phenyl-5-ethoxy-2-oxo-2,3-dihydroindole-1-sulphonyl]benzamide (Example 99)
N-tert-butyl-4-[3-methyl-3-(2-fluorophenyl)-5-ethoxy-2-oxo-2,3-dihydroindole-1-sulphonyl]benzamide (Example 101)
N-tert-butyl-4-[3-methyl-3-(2-chloro-6-fluorophenyl)-5-ethoxy-2-oxo-2,3-dihydro-indole-1-sulphonyl]benzamide (Example 125).

As used herein, the following definitions will apply unless otherwise stated:

“Patient” means a warm blooded animal, such as for example rat, mice, dogs, cats, guinea pigs, and primates such as humans.

“Treat” or “treating” means to alleviate symptoms, eliminate the causation of the symptoms either on a temporary or permanent basis, or to prevent or slow the appearance of symptoms of the named disorder or condition.

“Therapeutically effective amount” means a quantity of the compound which is effective in treating the named disorder or condition.

“Pharmaceutically acceptable carrier” is a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

The citation of any reference herein should not be construed as an admission that such reference is available as “Prior Art” to the instant application.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

According to one embodiment, the compounds of general formula (I) can be obtained according to Scheme 1 hereunder.

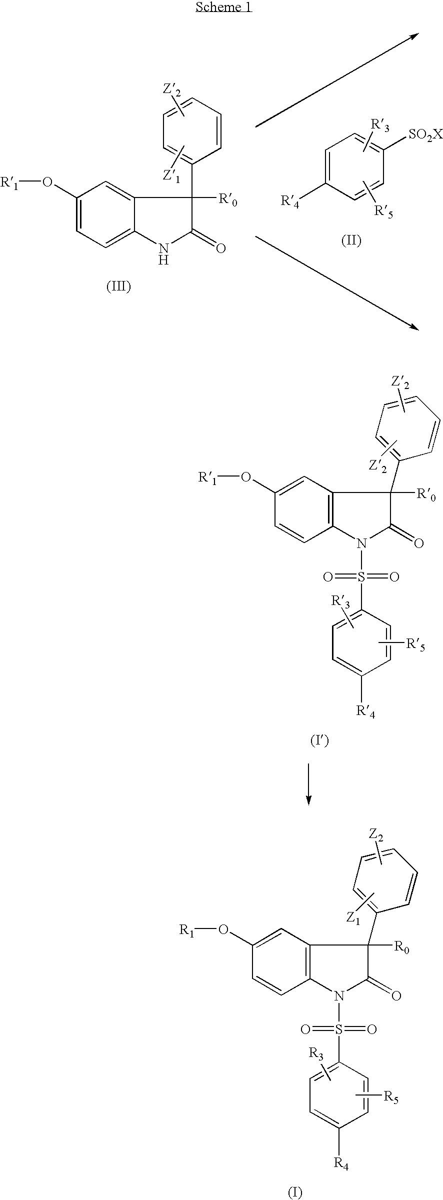

According to Scheme 1, the compounds of formula (I) are obtained by reaction of a compound of formula (II) with a compound of formula (III). In the compounds of formula (II), X represents a halogen atom and the groups R′₃, R′₄ and R′₅ represent respectively, and independently of one another, the groups R₃, R₄ and R₅ as defined for the compound of formula (I) or precursor groups of groups R₃, R₄ and R₅. In the compounds of formula (III), groups R′₀, R′₁, Z′₁ and Z′₂ represent respectively, and independently of one another, the groups R₀, R₁, Z₁ and Z₂ as defined for the compound of formula (I) or precursor groups of groups R₀, R₁, Z₁ and Z₂. The compounds of formula (I) are obtained directly by reaction of a compound of formula (II) with a compound of formula (III) when the groups R′₀, R′₁, R′₃, R′₄, R′₅, Z′₁ and Z′₂ represent respectively the groups R₀, R₁, R₃, R₄, R₅, Z₁ and Z₂ as defined for the compound of formula (I). Alternatively, the compounds of formula (I) are obtained indirectly via a compound of formula (I′) when at least one of the groups R′₀, R′₁, R′₃, R′₄, R′₅, Z′₁ and Z′₂ represents respectively a precursor group of the groups R′₀, R₁, R₃, R₄, R₅, Z₁ and Z₂ as defined for the compound of formula (I).

The addition of a compound of formula (II) to a compound of formula (III) is carried out in the presence of a metal hydride, for example sodium hydride, or of an alkaline alcoholate, for example potassium tert-butylate at temperatures between −40° and 25° C., in an anhydrous solvent such as tetrahydrofuran (THF).

The compounds of formula (I′) can also represent compounds of formula (I). The conversion of the precursor groups R′₀, R′₁, Z′₁, Z′₂, R′₃, R′₄ and R′₅ respectively, and independently of one another, to groups R′₀, R′₁, Z′₁, Z₂, R₃, R₄ and R₅ as defined, is achieved according to general, conventional techniques that are well known by a person skilled in the art, for example by reactions of alkylation, acylation, oxidation or reduction.

The compounds of formula (II) are commercially available, are known or are prepared according to methods known by a person skilled in the art. We may notably refer to the methods described in document WO98/25901 (page 20 lines 25 to 34 and page 21, lines 1 to 20).

In the invention it is also possible to prepare compounds of formula (II) in which R′₄ represents a group —C(O)OR_{c} or a group —C(O)NR₁₃R₁₄ by chemoselective reaction respectively of an alcohol H—OR_{c} or of an amine H—NR₁₃R₁₄ with a compound of formula (II) in which X represents a chlorine atom and R′₄ represents a group —C(O)Cl. This is carried out at temperatures between −30° C. and 0° C. in anhydrous solvents of low polarity such as ethers, or chlorinated solvents, for example dichloromethane.

The compounds of formula (III) can be prepared according to Scheme 2 hereunder.

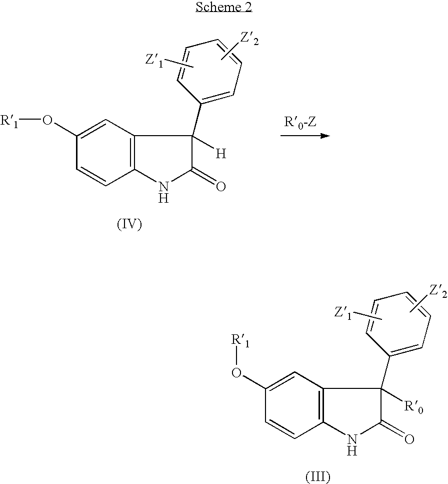

According to Scheme 2, the compounds of formula (III) can be prepared by the reaction of a compound of formula (IV) in which R′₁, Z′₁ and 12 are as defined for the compound of formula (III), in the presence of a base such as an alkaline alcoholate, potassium tert-butylate for example, with a derivative R′₀—Z in which R′₀ is as defined for the compound of formula (III) and Z represents a leaving group such as an iodine or a bromine or alternatively a sulphonate such as mesylate or tosylate, in an anhydrous solvent such as DMF or THF at temperatures between −40° C. and 20° C.

Alternatively, the compounds of formula (III) can be obtained according to Scheme 3 hereunder (Ph=phenyl).

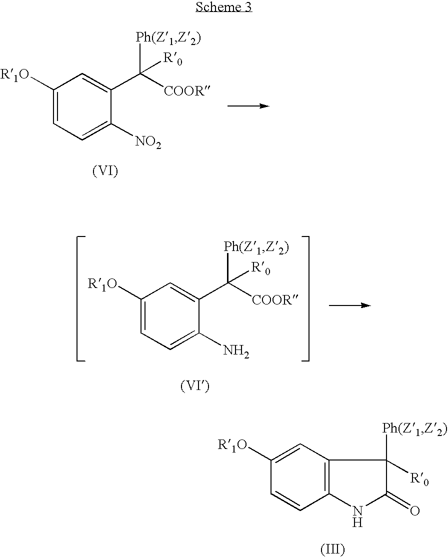

According to Scheme 3, the compounds of formula (III) can be obtained from the compounds of formula (VI)— in which R′₁, R′₀, Z′₁ and Z′₂ are as defined for the compounds of formula (III) and R″ represents a (C₁-C₃)alkyl group—by cyclization of the amine of formula (VI′)—in which R′₁, R′₀, Z′₁ and Z′₂ are as defined for the compounds of formula (III) and R″ represents a (C₁-C₃)alkyl group—generated in situ during reduction of the nitro group carried by the compound of formula (VI). This is carried out in the presence of a metal in an acid environment, such as tin or iron in an acid environment such as acetic acid at temperatures between 30 and 100° C.

The compounds of formula (IV) can be obtained according to Scheme 4 hereunder.

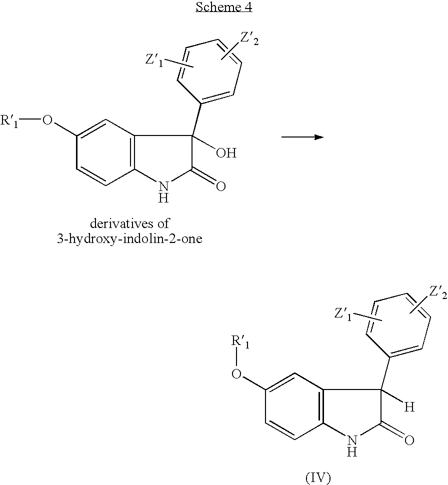

According to Scheme 4, the compounds of formula (IV) can be obtained by reduction of compounds derived from 3-hydroxy-indolin-2-one by reacting tin chloride in an acid environment, by analogy with the method described in Tetrahedron Letters 1996, 52 (20), 7003-7012 or Bioorganic and Medicinal Chemistry Letters 1997, 7 (10), 1255-1260. R′₁, Z′₁ and Z′₂ are as defined for the compounds of formula (III).

The 3-hydroxy-indolin-2-one derivatives are known or prepared from isatins that are commercially available or are known, by reaction with an organometallic derivative such as an organolithium or an organomagnesium compound according to, for example Biorg. Med. Lett. 7 (10); 1997; 1255.

The compounds of formula (IV) can also be obtained by the methods described notably in document WO01/74775 (page 19, lines 16 to 25 and page 20, lines 1, 2). We may mention for example the Brunner reaction described in Tetrahedron 1986; 42 (15), 4267-4272, the reaction of cyclization of mandelamide derivatives described in J. Org. Chem. 1968, 33 (4), 1640-1643, the cyclization reaction in the presence of formic acid described in J. Chem. Soc. Perkin Trans., 1986, 1, 349-360, and the cyclization reaction according to J. Am. Chem. Soc., 1985, 107 (2), 435-443.

The compounds of formula (IV) can also be prepared according to Scheme 5 hereunder.

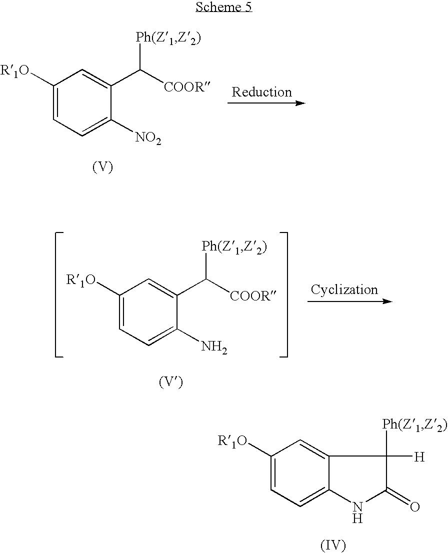

According to Scheme 5, we can also obtain the compounds of formula (IV)—in which R′₁, Z′₁ and Z′₂ are as defined for the compound of formula (III)—by cyclization of the amine (V′)—in which R′₁, Z′₁ and Z′₂ are as defined for the compound of formula (III) and R″ represents a (C₁-C₃)alkyl—generated in situ during reduction of the nitro group carried by the compound of formula (V)—in which R′₁, Z′₁ and Z₂ are as defined for the compound of formula (III) and R″ represents a (C₁-C₃)alkyl group. This is carried out in the presence of a metal in an acid environment, such as tin or iron in an acid environment such as acetic acid at temperatures between 30 and 100° C.

The compounds of formula (V) can be prepared according to Scheme 6 hereunder.

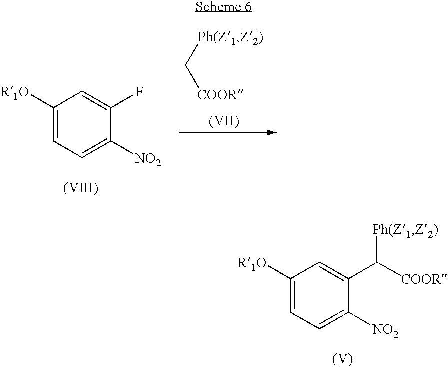

According to Scheme 6, the compounds of formula (V)—in which R′₁, Z′₁ and Z′₂ are as defined for the compound of general formula (III) and R″ represents a (C₁-C₃)alkyl group—can be obtained by a reaction of aromatic nucleophilic substitution of a nitrogen-containing ortho-halogeno compound of formula (VIII)—in which R′₁ is as defined for the compound of formula (III)—preferably ortho-fluoro and of an anion of a derivative of formula (VII)—in which Z′₁ and Z′₂ are as defined for the compound of formula (III) and R″ represents a (C₁-C₃)alkyl group—prepared by the action of a strong base such as an alkaline alcoholate, potassium tert-butylate or sodium hydride for example, in an anhydrous solvent such as DMF.

The compounds of formula (VI) can be prepared according to Scheme 7 hereunder.

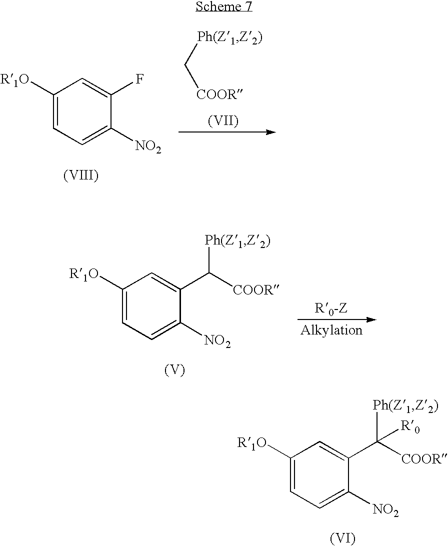

According to Scheme 7, we obtain the compounds of formula (VI)—in which R′₁, Z′₁, Z′₂, R′₀ are as defined for the compound of formula (III) and R″ represents a (C₁-C₃)alkyl group—by alkylation of the compound of formula (V) as defined previously for Scheme 6, in the presence of a base such as sodium hydride, with for example a derivative R′₀—Z in which R′₀ is as defined for the compound of formula (III) and Z represents a leaving group such as an iodine or a bromine or alternatively a sulphonate such as mesylate or tosylate, in an anhydrous solvent such as DMF or THF at temperatures between −40° C. and 20° C.

It is preferable not to isolate the compounds of formula (V) but submit them in situ to the derivatives R′₀—Z as defined previously to obtain the compounds of formula (VI).

The compounds of formula (VII) and (VIII) are commercially available, known or prepared according to methods well known by a person skilled in the art.

The N-oxides of the compounds bearing an amine are prepared according to methods known by a person skilled in the art by reaction of the amine with organic peracids such as peracetic, trifluoroperacetic, performic, perbenzoic acids or derivatives thereof such as 3-chloroperbenzoic acid, at temperatures between 0° C. and 90° C., preferably at temperatures below 50° C.

In the general synthesis schemes 1 to 7, the starting compounds and the reagents, when their method of preparation is not described, are commercially available or are described in the literature, or alternatively can be prepared according to methods that are described in the literature or are known by a person skilled in the art.

The pure enantiomers of the compounds of the invention can be prepared from enantiomerically pure precursors or alternatively by chiral phase chromatography or, when the compounds bear acid functions or amines, by selective crystallization of diastereoisomeric salts obtained by reaction of compounds (I) with, respectively, chiral amines or acids.

The preparations and examples that follow describe the preparation of certain compounds according to the invention. These preparations and examples are not limiting and are only intended to illustrate the present invention.

In the preparations and examples given below:
The mass spectra are recorded using a quadrupole spectrometer of the type Platform LCZ (WATERS) or of the type ZQ 4000 (WATERS) in positive electrospray ionization mode;
The NMR (nuclear magnetic resonance) spectra are recorded using a Fourier transform spectrometer (BRUKER), at a temperature of 300° K. (exchangeable protons not recorded);

s=singlet,

m=multiplet,

t=triplet,

q=quadruplet

DMSO-d₆=deuterated dimethylsulphoxide

CDCl₃=deuterated chloroform;

m.p.=melting point (in degrees Celsius) measured on a Kofler bench;

b.p.=boiling point (in degrees Celsius)

The rotatory power [α]D/20 is measured on a PERKIN-ELMER 241 polarimeter in standard conditions, at a temperature of 20° C.; the concentration c is expressed in g of solute per 100 ml of solution.

Mixtures of solvents are quantified in proportions by volume.

The microanalyses and NMR spectra confirm the structures of the compounds obtained according to the examples given above.

### PREPARATIONS (COMPOUNDS OF FORMULA (III)

### Preparation 1

### 3-(2-Chloro-phenyl)-5-ethoxy-3-methyl-1,3-dihydro-indol-2-one

### A) Preparation of 2-fluoro-4-ethoxy-nitrobenzene

Slowly add 229 g of caesium carbonate and then 65 ml of iodoethane to a solution of 100 g of 3-fluoro-4-nitrophenol in 2.5 L of acetonitrile. After stirring for 24 h at 50° C., treat with 2.4 L of water and 2.4 L ethyl acetate. After decanting, extract the aqueous phase again with 1 L of ethyl acetate. Dry the combined organic phases over sodium sulphate, and concentrate at reduced pressure. Take up the residue in pentane, filter, and dry. The expected product is obtained in the form of powder.

m. p.=48° C.

### B) 2-(2-Chloro-phenyl)-2-(5-ethoxy-2-nitro-phenyl)-methyl propionate

Add dropwise, at 0° C., a mixture of 16.15 g of 2-fluoro-4-ethoxy-nitrobenzene and 16.2 g of methyl 2-chlorophenyl acetate in solution in 200 ml of dimethylformamide to 10.5 g of sodium hydride at 60% in oil, suspended in 170 ml of dimethylformamide. Stir the reaction mixture for 3 hours, allowing the temperature to return to room temperature. Cool the reaction mixture to 0° C. then add 16.40 ml of iodomethane. Stir the reaction mixture at room temperature overnight. Add 17 ml of methanol and 500 ml of a saturated aqueous solution of sodium bicarbonate, and extract with 1000 ml of ethyl acetate. Wash the organic phase three times with 500 ml of a saturated aqueous solution of sodium bicarbonate, then three times with 500 ml of sodium chloride solution. Dry the organic phase over sodium sulphate, then evaporate the solvents at reduced pressure. The residue thus obtained is taken up in 50 ml of 2-propanol, stirred overnight, filtered, rinsed with pentane and dried at 50° C. under vacuum.

m.p.=89° C.

### C) 3-(2-Chloro-phenyl)-5-ethoxy-3-methyl-1,3-dihydro-indol-2-one

Add 19.9 g of iron then 28.5 ml of acetic acid to 25.9 g of 2-(2-chloro-phenyl)-2-(5-ethoxy-2-nitro-phenyl)-methyl propionate in suspension in 260 ml of ethanol. Heat the reaction mixture with stirring under reflux for 2 hours. Evaporate the solvents partially at reduced pressure then add 250 ml of a saturated aqueous solution of sodium bicarbonate and 600 ml of ethyl acetate. Stir the reaction mixture for 1 hour at room temperature, then filter and rinse with ethyl acetate. After decanting, wash the organic phase with 200 ml of a saturated aqueous solution of hydrogencarbonate, then with 200 ml of sodium chloride solution. Dry the organic phase over sodium sulphate and concentrate at reduced pressure. The residue thus obtained is taken up in pentane, filtered, and dried at 50° C. at reduced pressure.

m.p.=140° C.

¹H NMR 250 MHz (DMSO-d₆): 1.21 (t, 3H); 1.65 (s, 3H); 3.88 (q, 2H); 6.34 (s, 1H); 6.71-6.82 (m, 2H); 7.3-7.5 (m, 3H); 7.75-7.8 (m, 1H)

### D) Enantiomeric Resolution

Enantiomeric resolution of the preceding compound prepared in C) is carried out by supercritical chiral-phase chromatography in the following conditions:

Equipment: Berger Prep SFC supercritical chromatography system with Pronto software.

Chiral column: CHIRALPAK AD-H 5 μm, Length: 25 cm, diameter: 21 mm

Mobile phase: CO₂/Methanol

Flow: 50 ml/min

Pressure: 100 bar

UV detection: 220 nm

We thus obtain laevorotatory 3-(2-chloro-phenyl)-5-ethoxy-3-methyl-1,3-dihydro-indol-2-one:

m.p.=80° C.

[α]D/20=-7.1° (c=0.5 in EtOAc) as well as its dextrorotatory enantiomer.

### Preparation 2

### 4-Chloro-3-(5-ethoxy-3-methyl-2-oxo-2,3-dihydro-1H-indol-3-yl)-benzoic acid

### A) 4-Chloro-3-(1-methoxycarbonyl-ethyl)-methyl benzoate

### A1) 4-chloro-3-methyl-methyl benzoate

Add 50 g of caesium carbonate and 10.95 ml of methyl iodide dropwise to a solution of 25 g of 4-chloro-3-methylbenzoic acid in 550 ml of dimethylformamide. Stir the reaction mixture at room temperature for 16 h, then add 800 ml of an aqueous solution of sodium bicarbonate, and extract with ethyl acetate. After decanting, extract the aqueous phase again with 400 ml of ethyl acetate. Wash the combined organic phases with 250 ml of an aqueous solution of sodium bicarbonate and then with 250 ml of an aqueous solution of sodium chloride. Dry the organic phase obtained over sodium sulphate and concentrate at reduced pressure. The desired product is purified by distillation at reduced pressure, and an oil is obtained.

b.p.=78° C./0.08 mbar

### A2) 3-bromomethyl-4-chloromethyl benzoate

Add 24.85 g of N-bromosuccinimide to a solution of 25.26 g of 4-chloro-3-methyl-methyl benzoate in 20 ml of carbon tetrachloride. Stir the reaction mixture under reflux for 6 h, then add 500 ml of dichloromethane and 300 ml of an aqueous solution of potassium carbonate at 25° C. After decanting, extract the aqueous phase again with 200 ml of dichloromethane. Wash the combined organic phases with 300 ml of an aqueous solution of potassium carbonate, dry over sodium sulphate and concentrate at reduced pressure. The desired product, purified by distillation at reduced pressure, crystallizes at room temperature.

m.p.=93° C.

### A3) 4-chloro-3-cyanomethyl-methyl benzoate

Add a solution of 7.12 g of sodium cyanide in 100 ml of water at 10° C. to a solution of 25.52 g of 3-bromomethyl-4-chloromethyl benzoate in 170 ml of 1,4-dioxan. Stir the reaction mixture at room temperature for 16 h, then add 500 ml of an aqueous solution of potassium carbonate and 600 ml of ethyl acetate. After decanting, extract the aqueous phase again with 250 ml of ethyl acetate. Wash the combined organic phases with 300 ml of an aqueous solution of potassium carbonate, dry over sodium sulphate and concentrate at reduced pressure.

The expected product is obtained in the form of powder.

m.p.=87° C.

### A4) 4-chloro-3-(1-methoxycarbonyl-ethyl)-methyl benzoate

Add gaseous anhydrous hydrochloric acid for 3 h at 0° C. to a solution of 19.68 g of 4-chloro-3-cyanomethyl-methyl benzoate in 207 ml of methanol.

Stir the reaction mixture for 16 h at room temperature, then concentrate at reduced pressure. Take up the residue in 600 ml of ethyl acetate and 500 ml of water. Wash the organic phase with 200 ml of an aqueous solution of sodium chloride, dry over sodium sulphate and concentrate at reduced pressure.

The expected product is obtained in the form of crystals.

m.p.=55° C.

### B) 4-Chloro-3-[1-(5-ethoxy-2-nitro-phenyl)-1-methoxycarbonyl-ethyl]-methyl benzoate

Add, at −5° C., a mixture of 30.5 g of 2-fluoro-4-ethoxy-nitrobenzene (Preparation 1A) and 39.97 g of 4-chloro-3-(1-methoxycarbonyl-ethyl)-methyl benzoate dissolved in 200 ml of dimethylformamide to 19.76 g of a dispersion of sodium hydride at 60% in oil, in 300 ml of dimethylformamide. Stir the reaction mixture at room temperature for 2.5 h, then add 30.7 ml of methyl iodide, at 10° C. Stir the reaction mixture at room temperature for 16 h then add 30 ml of methanol at 10° C., as well as dilute aqueous solution of sodium bicarbonate. Extract with ethyl acetate, dry the organic phase over sodium sulphate and evaporate the solvents at reduced pressure.

The expected product is obtained in the form of powder.

m.p.=141° C.

### C) 4-Chloro-3-(5-ethoxy-3-methyl-2-oxo-2,3-dihydro-1H-indol-3-yl)-methyl benzoate

Add 41.49 g of iron and 59.49 ml of acetic acid to a solution of 4-chloro-3-[1-(5-ethoxy-2-nitro-phenyl)-1-methoxycarbonyl-ethyl]-methyl benzoate in 650 ml of ethanol. After stirring for 5 h under reflux, concentrate the reaction mixture at reduced pressure, add 700 ml of dilute aqueous solution of sodium bicarbonate and extract with 1400 ml of ethyl acetate. Stir the reaction mixture for 1 hour at room temperature, then filter and rinse with ethyl acetate.

After decanting, wash the organic phase with 600 ml of dilute aqueous solution of sodium bicarbonate, then with 600 ml of sodium chloride solution. Dry the organic phase over sodium sulphate and concentrate at reduced pressure. Take up the residue in pentane, filter, and dry.

The expected product is obtained in the form of powder.

m.p.=176° C.

¹H NMR 250 MHz (DMSO-d₆): 1.2 (t, 3H); 1.7 (s, 3H); 3.79-3.88 (m, 5H); 6.4/6.43 (m, 1H); 6.71-6.82 (m, 2H); 7.5-7.55 (m, 1H); 7.88-7.97 (m, 1H); 8.25-8.9 (m, 1H).

### D) 4-Chloro-3-(5-ethoxy-3-methyl-2-oxo-2,3-dihydro-1H-indol-3-yl)-benzoic acid

Add 90 ml of 2N sodium hydroxide solution to a solution of 18 g of 4-chloro-3-(5-ethoxy-3-methyl-2-oxo-2,3-dihydro-1H-indol-3-yl)-methyl benzoate in 90 ml of methanol and 180 ml of dioxan. After stirring for 16 h at room temperature, concentrate the reaction mixture at reduced pressure.

Cool the reaction mixture to a temperature of about 0° C. Add 500 ml of water and 180 ml of a 1N aqueous hydrochloric acid solution. Filter the precipitate and then rinse with water. The residue obtained is dried under vacuum.

The expected product is obtained in the form of powder.

m.p.=234° C.

### E) Enantiomeric Resolution

Enantiomeric resolution of the preceding acid is performed by supercritical chiral-phase chromatography in the following conditions:

Equipment: Berger Prep SFC system for supercritical chromatography with Pronto software

Chiral column: CHIRALPAK AD-H 5 μm, Length: 25 cm, diameter: 21 mm

Mobile phase: CO₂/Methanol (60%140%)

Flow: 50 ml/min

Pressure: 100 bar

UV detection: 220 nm

We thus obtain dextrorotatory 4-chloro-3-(5-ethoxy-3-methyl-2-oxo-2,3-dihydro-1H-indol-3-yl)-benzoic acid:

m.p.=236° C.

¹H NMR 400 MHz (DMSO-d₆): 1.22 (t, 3H); 1.69 (s, 3H); 3.8-3.9 (m, 2H); 6.38-6.42 (m, 1H); 6.71-6.82 (m, 2H); 7.45-7.51 (m, 1H); 7.85-7.92 (m, 1H); 8.28 (s, 1H)

[α]D/20=+125° (c=1, in ethyl acetate) as well as its laevorotatory enantiomer.

### Preparation 3

### 3-(5-Amino-2-chloro-phenyl)-5-ethoxy-3-methyl-1,3-dihydro-indol-2-one

### A) (2-Chloro-5-nitro-phenyl)-methyl acetate

Using a diffusing plunger tube, dissolve hydrogen chloride in a solution of 28 g of (2-chloro-5-nitrophenyl)-acetonitrile [described by Lisitsyn, V. N.; Lugovskaya in J. Org. Chem. USSR (Engl. Transl.); EN; 10; 1974; 92-95] in 300 ml of methanol, cooled to 0° C., until there is saturation at 0° C. Continue stirring under a gentle stream of nitrogen at 20° C. for 15 h. After concentration at reduced pressure, take up in 500 ml of ethyl acetate and 500 ml of a mixture of water and ice. Dry the organic phase over sodium sulphate, and concentrate at reduced pressure. The residue is purified by chromatography on a column of 400 g of silica, eluting with a cyclohexane/ethyl acetate mixture. The expected product is obtained in liquid form.

¹H NMR 250 MHz (CDCl3): 3.79 (s, 3H); 3.92 (s, 2H); 7.61 (d, 1H); 8.1-8.3 (m, 2H).

### B) 2-(2-Nitro-5-ethoxy-phenyl)-2-(2-chloro-5-nitro-phenyl)-methyl propionate

Add, dropwise, a solution of 18.3 g of (2-chloro-5-nitro-phenyl)-methyl acetate and 14.76 g of 2-fluoro-4-ethoxy-nitrobenzene (Preparation 1A) in 160 ml of dimethylformamide to a suspension of 12.4 g of sodium hydride in 200 ml of dimethylformamide, cooled to −10° C. and under a nitrogen atmosphere. Stir for 4 h, allowing the temperature to return to 20° C. At 0° C. add, dropwise, 15 ml of methyl iodide and then stir for 16 h at 20° C. At 10° C., treat with 20 ml of methanol, 1.5 L of aqueous solution of sodium bicarbonate and 1.5 L of ethyl acetate. After decanting, wash the organic phase with 2×1.5 L of aqueous solution of sodium bicarbonate, dry over sodium sulphate, and concentrate at reduced pressure. Crystallize the residue from toluene, filter, and dry. The expected product is obtained in the form of powder.

m.p.=195° C.

C) 3-(5-Amino-2-chloro-phenyl)-5-ethoxy-3-methyl-1,3-dihydro-indol-2-one

Add 33 g of iron to a solution of 32.6 g of 2-(2-amino-5-ethoxy-phenyl)-2-(2-chloro-5-nitro-phenyl)-methyl propionate in 900 ml of methanol and 70 ml of acetic acid. After stirring for 3 h under reflux and overnight at 20° C., concentrate the reaction mixture at reduced pressure. Add 1 L of dilute aqueous solution of sodium bicarbonate and extract with 0.8 L of ethyl acetate. Stir the reaction mixture for 1 hour at 20° C., then filter and rinse with ethyl acetate. After decanting, wash the organic phase with 0.5 L of dilute aqueous solution of sodium bicarbonate, then with 0.5 L of sodium chloride solution. Dry the organic phase over sodium sulphate and concentrate at reduced pressure. Crystallize the residue from a toluene/2-propanol mixture, filter, and dry.

The expected product is obtained in the form of a beige powder.

m.p.=166° C.

¹H NMR 250 MHz (DMSO-d₆): 1.23 (t, 3H); 1.54 (s, 3H); 3.88 (q, 2H); 6.31-6.38 (m, 1H); 6.48-6.51 (m, 1H); 6.89-6.99 (m, 2H); 7.15-7.3 (m, 2H)

### D) Enantiomeric Resolution

Enantiomeric resolution of the preceding compound prepared in C) is performed by supercritical chiral-phase chromatography in the following conditions:

Equipment: Berger Prep SFC system for supercritical chromatography with Pronto software

Chiral column: CHIRALPAK AD-H 5 μm, Length: 25 cm, diameter: 21 mm

Mobile phase: CO₂/Methanol (60%/40%)

Flow: 50 ml/min

Pressure: 100 bar

UV detection: 220 nm

We thus obtain laevorotatory 3-(5-amino-2-chloro-phenyl)-5-ethoxy-3-methyl-1,3-dihydro-indol-2-one:

m.p.=172° C.

[α]/D/20=-9.7° (c=1 in MeOH)] as well as its dextrorotatory enantiomer.

### Preparation 4

### 4-Chloro-3-(5-ethoxy-3-methyl-2-oxo-2,3-dihydro-1H-indol-3-yl)-benzaldehyde

### A) Preparation of laevorotatory 4-chloro-3-(5-ethoxy-3-methyl-2-oxo-2,3-dihydro-1H indol-3-yl)-methyl benzoate

Dissolve 4 g of laevorotatory 4-chloro-3-(5-ethoxy-3-methyl-2-oxo-2,3-dihydro-1H-indol-3-yl)-benzoic acid obtained in Preparation 2, in 50 ml of DMF. Add 2.35 g of potassium carbonate and then 1.08 ml of iodomethane. After stirring overnight at 20° C., pour into a water-ice mixture and extract with ethyl acetate. Dry the organic phase over sodium sulphate, evaporate to dryness and then purify by silica chromatography to obtain the desired compound in the form of powder.

m.p.=84° C.

[α]D/20=−91.9° (c=1 in MeOH)

¹H NMR 250 MHz (DMSO-d₆): 1.21 (t, 3H); 1.69 (s, 3H); 3.79-3.81 (m, 2H); 3.92 (s, 3H); 6.43 (s, 1H); 6.71-6.81 (m, 2H); 7.49-7.54 (m, 1H); 7.89-7.92 (m, 1H); 8.28 (s, 1H)

### B) Preparation of 3-(2-chloro-5-hydroxymethyl-phenyl)-5-ethoxy-3-methyl-1,3-dihydro-indol-2-one

Dissolve 2 g of the preceding ester in 40 ml of dichloromethane. After cooling to −70° C., slowly add 7.48 ml of diisobutylaluminium hydride 1.5M in toluene. After stirring for 2 h at 20° C., add 3.7 ml of diisobutylaluminium hydride at −70° C., then stir overnight at 20° C. Carry out partial evaporation of the solvent at reduced pressure. Take up in 175 ml of 1N hydrochloric acid, then extract with ethyl acetate. Dry the organic phase over sodium sulphate, evaporate to dryness, then purify by silica chromatography to obtain the desired compound in the form of powder.

Mass spectometry: MS[(+)ESI, m/z]: 331 (MH+)

¹H NMR 250 MHz (DMSO-d₆): 1.2 (t, 3H); 1.62 (s, 3H); 3.85 (q, 2H); 4.9 (d. 2H); 6.31-6.36 (m, 1H); 6.7-6.81 (m, 2H); 7.28-7.32 (m, 2H); 7.69-7.71 (m, 1H)

MS[(+)ESI, m/z]: 332 (MH+)

### C) Preparation of 4-chloro-3-(5-ethoxy-3-methyl-2-oxo-2,3-dihydro-1H-indol-3-yl)-benzaldehyde

Add 1.89 g of pyridinium dichromate, at 0° C., to 1.67 g of the preceding alcohol dissolved in 22 ml of dichloromethane. After stirring overnight at 20° C., filter on celite, rinse with 50 ml of dichloromethane, evaporate the filtrate and then purify by silica chromatography to obtain the desired compound in the form of powder.

¹H NMR 250 MHz (DMSO-d₆): 1.2 (t, 3H); 1.74 (s, 3H); 3.85 (q, 2H); 6.4-6.48 (m, 1H); 6.7-6.86 (m, 2H); 7.58-7.65 (m, 1H); 7.85-7.95 (m, 1H); 8.3 (m, 1H); 10.1 (s, 1H)

MS[(+)ESI, m/z]: 330 (MH+)

### Preparation 5

### 3-(2-Fluoro-phenyl)-5-ethoxy-3-methyl-1,3-dihydro-indol-2-one

### A) 2-(2-Fluoro-phenyl)-2-(5-ethoxy-2-nitro-phenyl)-methyl propionate

2-(2-Fluoro-phenyl)-2-(5-ethoxy-2-nitro-phenyl)-methyl propionate is obtained in the same way as for Preparation 1B from 2-fluorophenylmethyl acetate.

¹H NMR 250 MHz (DMSO): 1.28 (t, 3H); 2.07 (s, 3H); 3.57 (s, 3H); 3.95-4.1 (m, 2H); 6.55-6.58 (m, 1H); 7.07-7.31 (m, 3H); 7.4-7.49 (m, 1H); 7.56-7.64 (m, 1H); 7.97-8.02 (m, 1H)

### B) 3-(2-Fluoro-phenyl)-5-ethoxy-3-methyl-1,3-dihydro-indol-2-one

3-(2-Fluoro-phenyl)-5-ethoxy-3-methyl-1,3-dihydro-indol-2-one is obtained in the same way as for Preparation 1C from the preceding compound.

¹H NMR 250 MHz (DMSO-d₆): 1.23 (t, 3H); 1.64 (s, 3H); 3.8-3.92 (m, 2H); 6.48-6.51 (m, 1H); 6.71-6.82 (m, 2H); 7.01-7.11 (m, 1H); 7.26-7.41 (m, 2H); 7.61-7.7 (m, 1H)

### C) Enantiomeric Resolution

Enantiomeric resolution is performed in the same way as for Preparation 1D, from the preceding compound.

We thus obtain dextrorotatory 3-(2-fluoro-phenyl)-5-ethoxy-3-methyl-1,3-dihydro-indol-2-one:
[α]D/20=+9.6° (c=1 in EtOAc) as well as its laevorotatory enantiomer.

### Preparation 6

### 3-Phenyl-5-ethoxy-3-methyl-1,3-dihydro-indol-2-one

### A) 2-Phenyl-2-(5-ethoxy-2-nitro-phenyl)-methyl propionate

2-Phenyl-2-(5-ethoxy-2-nitro-phenyl)-methyl propionate is obtained in the same way as for Preparation 1B from phenyl methyl acetate.

¹H NMR 250 MHz (DMSO-d₆): 1.24 (t, 3H); 2.07 (s, 3H); 3.53 (s, 3H); 3.92-4.1 (m, 2H); 6.26-6.29 (m, 1H); 7.03-7.09 (m, 1H); 7.35-7.48 (m, 5H); 8.03-8.08 (m, 1H)

### B) 3-Phenyl-5-ethoxy-3-methyl-1,3-dihydro-indol-2-one

3-Phenyl-5-ethoxy-3-methyl-1,3-dihydro-indol-2-one is obtained in the same way as for Preparation 1C from the preceding compound.

¹H NMR 250 MHz (DMSO-d₆): 1.27 (t, 3H); 1.66 (s, 3H); 3.87-3.98 (m, 2H); 6.75-6.87 (m, 3H); 7.20-7.36 (m, 5H)

### C) 3-Phenyl-5-ethoxy-3-methyl-1,3-dihydro-indol-2-one laevorotatory isomer and dextrorotatory isomer obtained by enantiomeric resolution

Enantiomeric resolution is performed in the same way as for Preparation 1D from the preceding compound.

We thus obtain 3-phenyl-5-ethoxy-3-methyl-1,3-dihydro-indol-2-one:
Dextrorotatory

[α]D/20=+98° (c=1 in MeOH).

H¹ NMR 250 MHz (DMSO-d₆): 1.28 (t, 3H); 1.66 (s, 3H); 3.88-3.98 (m, 2H); 6.75-6.87 (m, 3H); 7.21-7.37 (m, 5H)
Laevorotatory

[α]D/20=-110° (c=1 in MeOH)

In similar conditions to steps A, B and C described above, the compounds of the preparations collated in Table I below are obtained, starting from 2-fluoro-4-ethoxy-nitrobenzene (Preparation 1A) and suitable, commercially available or described, variously substituted phenyl methyl acetates or phenyl ethyl acetates.

TABLE I

| | | | | | | | (III) |
|---|---|---|---|---|---|---|---|
| 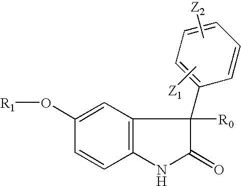 | | | | | | | |
| | | | | | | m.p. | |
| Preparation | R₀ | R₁ | Z₁ | Z₂ | [α]D/20 | (° C.) | MH+ |
| 7 dextrorotatory | Me | Et | 3-OMe | H | +154° (c = 1, EtOAc) | 109 | 298 |
| 7 laevorotatory | Me | Et | 3-OMe | H | −163° (c = 1, EtOAc) | 101 | 298 |
| 8 dextrorotatory | Me | Et | 2-OMe | H | +26° (c = 1, EtOAc) | 152 | 298 |
| 8 laevorotatory | Me | Et | 2-OMe | H | −24° (c = 1, EtOAc) | 152 | 298 |
| 9 dextrorotatory | Me | Et | 4-Me | H | +189° (c = 1, EtOAc) | 104 | 282 |
| 9 laevorotatory | Me | Et | 4-Me | H | −186° (c = 1, EtOAc) | 104 | 282 |
| 10 dextrorotatory | Me | Et | 3-Me | H | +163° (c = 1, EtOAc) | 108 | 282 |
| 10 laevorotatory | Me | Et | 3-Me | H | −163° (c = 1, EtOAc) | 107 | 282 |
| 11 dextrorotatory | Me | Et | 2-OCF3 | H | +49° (c = 1, EtOAc) | 120 | 352 |
| 11 laevorotatory | Me | Et | 2-OCF3 | H | −49° (c = 1, EtOAc) | 120 | 352 |
| 12 dextrorotatory | Me | Et | 2-CF3 | H | +432° (c = 1, EtOAc) | 152 | 336 |
| 12 laevorotatory | Me | Et | 2-CF3 | H | −419° (c = 1, EtOAc) | 181 | 336 |
| 13 dextrorotatory | Me | Et | 2-Cl | 3-Cl | +51° (c = 1, EtOAc) | 192 | 336 |
| 13 laevorotatory | Me | Et | 2-Cl | 3-Cl | −51° (c = 1, EtOAc) | 191 | 336 |
| 14 dextrorotatory | Me | Et | 2-Cl | 6-F | +75° (c = 1, EtOAc) | 139 | 320 |
| 14 laevorotatory | Me | Et | 2-Cl | 6-F | −80° (c = 1, EtOAc) | 139 | 320 |
| 15 dextrorotatory | Me | Et | 2-Cl | 5-OMe | +43 (c = 1, MeOH) | 87 | 332 |
| 15 laevorotatory | Me | Et | 2-Cl | 5-OMe | −71 (c = 1, MeOH) | 85 | 332 |
| 16 dextrorotatory | Me | Et | 2-OBn | H | +66° (c = 1, EtOAc) | 100 | 374 |
| 16 laevorotatory | Me | Et | 2-OBn | H | −58° (c = 1, EtOAc) | 92 | 374 |
| 17 dextrorotatory | Me | Et | 4-OBn | H | +161° (c = 1, EtOAc) | 147 | 374 |
| 17 laevorotatory | Me | Et | 4-OBn | H | −164° (c = 1, EtOAc) | 148 | 374 |

### Preparation 18

### 3-(2-Chlorophenyl)-5-ethoxy-3-ethyl-1,3-dihydroindol-2-one

### A) Methyl 2-(2-chlorophenyl)-2-(5-ethoxy-2-nitrophenyl)butyrate

Add, at 0° C., dropwise, the mixture of 10 g of 2-fluoro-4-ethoxynitrobenzene prepared in A of preparation 1 and 11.96 g of methyl 2-chlorophenyl acetate in solution in 60 ml of dimethylformamide to 7.07 g of sodium hydride at 60% in oil in suspension in 160 ml of dimethylformamide. Stir the reaction mixture for 3 hours, allowing the temperature to return to room temperature. Cool the reaction mixture to 0° C., then add 33.7 g of iodo-ethane. Stir the reaction mixture at room temperature overnight. Add 17 ml of methanol and 500 ml of a saturated aqueous solution of sodium hydrogen carbonate, and extract with 1000 ml of ethyl acetate. Wash the organic phase three times with 500 ml of a saturated aqueous solution of sodium hydrogen carbonate, then three times with 500 ml of sodium chloride solution. Dry the organic phase over sodium sulphate, then evaporate the solvents at reduced pressure. The residue thus obtained is taken up with 50 ml of 2-propanol, stirred overnight, filtered, rinsed with pentane and dried at 50° C. under vacuum.

m.p.=95° C.

### B) 3-(2-Chlorophenyl)-5-ethoxy-3-ethyl-1,3-dihydroindol-2-one

Add 14.5 g of iron then 21.03 ml of acetic acid to 19.6 g of methyl 2-(2-chlorophenyl)-2-(5-ethoxy-2-nitrophenyl)butyrate in suspension in 208 ml of methanol. Heat the reaction mixture with stirring under reflux for 2 hours. Evaporate the solvents partially at reduced pressure then add 250 ml of a saturated aqueous solution of sodium hydrogen carbonate and 600 ml of ethyl acetate. Stir the reaction mixture for 1 hour at room temperature, then filter and rinse with ethyl acetate. After decanting, wash the organic phase with 200 ml of a saturated aqueous solution of hydrogen carbonate, then with 200 ml of sodium chloride solution. Dry the organic phase over sodium sulphate and concentrate at reduced pressure. The residue thus obtained is taken up with pentane, filtered, and dried at 50° C. at reduced pressure.

m.p.=157° C.

### C) Enantiomeric Resolution

Enantiomeric resolution of the 3-(2-chlorophenyl)-5-ethoxy-3-ethyl-1,3-dihydroindol-2-one is carried out by supercritical chiral-phase chromatography under the following conditions: Equipment: Berger Prep SFC supercritical chromatography system with Pronto software. Chiral column: CHIRALPAK AD-H 5 μm, length: 25 cm, diameter: 21 mm

Mobile phase: CO₂/methanol

Flow rate: 50 ml/min

Pressure: 100 bar

UV detection: 220 nm

We thus obtain laevorotatory 3-(2-chlorophenyl)-5-ethoxy-3-ethyl-1,3-dihydroindol-2-one:

m.p.=89° C.

[α]D/20=−34.6° (c=1 in EtOAc) and also its dextrorotatory enantiomer.

### Preparation 19

### 3-(2-Chlorophenyl)-5-ethoxy-3-propyl-1,3-dihydroindol-2-one

Under conditions similar to preparation 18, replacing the iodoethane with iodopropane in stage A, laevorotatory 3-(2-chlorophenyl)-5-ethoxy-3-propyl-1,3-dihydroindol-2-one is obtained:

m.p.=136° C.

[α]D/20=−43.1° (c=1 in EtOAc) and also its dextrorotatory enantiomer.

### EXAMPLES

In Tables II to XI given below, Me denotes a methyl group, Et denotes an ethyl group, Pr denotes an n-propyl group and Bn denotes a benzyl group.

### Example 1

### 3-(2-Chloro-phenyl)-5-ethoxy-1-(2,4-dimethoxy-benzenesulphonyl)-3-methyl-1,3-dihydro-indol-2-one

Add 0.123 g of potassium tert-butylate to a solution of 0.3 g of laevorotatory 3-(2-chloro-phenyl)-5-ethoxy-3-methyl-1,3-dihydro-indol-2-one (Preparation 1) in 7 ml of tetrahydrofuran cooled to −30° C. Allow the temperature to return to 0° C., cool to −60° C. and then add 0.259 g of 2,4-dimethoxy-benzenesulphonyl chloride. After stirring overnight at 20° C., hydrolyse with water and extract with ethyl acetate. Dry the organic phase over sodium sulphate, and evaporate to dryness. Take up the residue in isopropyl ether while stirring. The expected product, in the form of white powder, is filtered and dried under vacuum.

m.p.=197° C.

¹H NMR 250 MHz (DMSO-d₆): 1.25 (t, 3H); 1.68 (s, 3H); 3.61 (s, 3H); 3.8-3.99 (m, 5H); 6.33 (d. 1H); 6.69-6.78 (m, 2H); 6.9-6.98 (m, 1H); 7.28-7.5 (m, 3H); 7.7-7.81 (m, 2H); 7.89-7.92 (m, 1H)

### Example 2

### N-tert-Butyl-4-[3-(2-chloro-phenyl)-5-ethoxy-3-methyl-2-oxo-2,3-dihydro-indole-1-sulphonyl]-benzamide

### A) Preparation of 4-tert-butylcarbamoyl-benzenesulphonyl chloride

### a) 4-Chlorosulphonyl-benzoyl chloride

Add 33 ml of sulphonyl chloride to a solution of 25 g of 4-chlorosulphonyl-benzoic acid in 250 ml of toluene. Heat under reflux for 5 h. At a temperature of 40° C. at reduced pressure, evaporate the solvents and take up the residue in 100 ml of heptane. By filtration and rinsing with a small amount of heptane, the desired product is obtained in the form of white powder.

¹H NMR 250 MHz (CDCl₃): 8.21 (m, 2H); 8.41 (m, 2H)

### b) 4-tert-Butylcarbamoyl-benzenesulphonyl chloride

Add, very slowly, 15 g of tert-butylamine diluted in 70 ml of dichloromethane to a solution of 24.5 g of the preceding compound in 250 ml of dichloromethane cooled to 0° C. Filter on a frit, rinse with dichloromethane, and dry under vacuum at 40° C. to obtain the expected product in the form of a white powder.

m.p.=179° C.

¹H NMR 250 MHz (CDCl₃): 1.51 (s, 9H); 7.92-8 (m, 2H); 8.09-8.16 (m, 2H)

### B) N-tert-Butyl-4-[3-(2-chloro-phenyl)-5-ethoxy-3-methyl-2-oxo-2,3-dihydro-indole-1-sulphonyl]-benzamide

Add 0.0614 g of potassium tert-butylate to a solution of 0.15 g of laevorotatory 3-(2-chloro-phenyl)-5-ethoxy-3-methyl-1,3-dihydro-indol-2-one (Preparation 1) in 4 ml of tetrahydrofuran cooled to −30° C. Allow the temperature to return to 0° C., then cool to −60° C. and add 0.15 g of 4-tert-butylcarbamoyl-benzenesulphonyl chloride. After stirring overnight at 20° C., hydrolyse with water and extract with ethyl acetate. Dry the organic phase over sodium sulphate, evaporate to dryness and purify by silica chromatography eluting with a cyclohexane/ethyl acetate mixture 6/4. The desired product is obtained in the form of a white powder.

m.p.=157° C.

¹H NMR 400 MHz (DMSO-d₆): 1.22 (t, 3H); 1.38 (s, 9H); 1.71 (s, 3H); 3.84-3.94 (m, 2H); 6.92 (m, 1H); 7.28 (m, 1H); 7.35 (m, 1H); 7.45 (m, 1H); 7.75-7.81 (m, 2H); 7.99-8.11 (m, 5H)

### Example 3

### 4-[3-(2-Chloro-phenyl)-5-ethoxy-3-methyl-2-oxo-2,3-dihydro-indole-1-sulphonyl]-N-cyclopentyl-benzamide

### A) Preparation of 4-cyclopentylcarbamoyl-benzenesulphonyl chloride

Add, very slowly, 0.83 ml of cyclopentylamine to a solution of 1 g of 4-chlorosulphonyl-benzoyl chloride (Example 2Aa) in 15 ml of dichloromethane cooled to 0° C. After stirring for 3 h at 20° C., wash with water, dry over sodium sulphate, evaporate to dryness, and purify by silica chromatography eluting with a cyclohexane/ethyl acetate mixture in the proportions 90/10, respectively. The desired product is obtained in the form of a white powder.

m.p.=143° C.

¹H NMR 250 MHz (CDCl3): 1.47-1.64 (m, 2H); 1.69-1.89 (m, 4H); 2.08-2.26 (m, 2H); 4.38-4.54 (m, 1H); 7.95-8.03 (m, 2H); 8.1-8.18 (m, 2H)

### B) 4-[3-(2-Chloro-phenyl)-5-ethoxy-3-methyl-2-oxo-2,3-dihydro-indole-1-sulphonyl]-N-cyclopentyl-benzamide

4-[3-(2-Chloro-phenyl)-5-ethoxy-3-methyl-2-oxo-2,3-dihydro-indole-1-sulphonyl]-N-cyclo-pentyl-benzamide is obtained in a similar way to Example 2, from laevorotatory 3-(2-chloro-phenyl)-5-ethoxy-3-methyl-1,3-dihydro-indol-2-one (Preparation 1) and sulphonyl chloride is prepared according to stage A of Example 3.

m.p.=152° C.

¹H NMR 250 MHz (DMSO-d₆): 1.29 (t, 3H); 1.5-2 (m, 11H); 3.85-3.98 (m, 2H); 4.18-4.3 (m, 1H); 6.91-6.99 (m, 1H); 7.22-7.28 (m, 1H); 7.3-7.49 (m, 2H); 7.75-7.85 (m, 2H); 8.05-8.18 (m, 4H); 8.57-8.63 (m, 1H)

### Example 4

### 4-[3-(2-Chloro-phenyl)-5-ethoxy-3-methyl-2-oxo-2,3-dihydro-indole-1-sulphonyl]-N-(1,1-dimethyl-propyl)-benzamide

### A) Preparation of 4-(1,1-dimethyl-propyl)-carbamoyl-benzenesulphonyl chloride

Add, very slowly, 0.98 ml of 1,1-dimethyl-propylamine to a solution of 1 g of 4-chlorosulphonyl-benzoyl chloride (Example 2Aa) in 15 ml of dichloromethane cooled to 0° C. After stirring for 3 h at 20° C., wash with water, dry over sodium sulphate, evaporate to dryness, and purify by silica chromatography, eluting with a cyclohexane/ethyl acetate mixture in the proportions 90/10, respectively. The desired product is obtained in the form of a white powder.

m.p.=126° C.

¹H NMR 250 MHz (CDCl3): 0.95 (t, 3H); 1.47 (s, 6H); 1.9 (q, 2H); 7.93-7.98 (m, 2H); 8.1-8.16 (m, 2H)

### B) 4-[3-(2-Chloro-phenyl)-5-ethoxy-3-methyl-2-oxo-2,3-dihydro-indole-1-sulphonyl]-N-(1,1-dimethyl-propyl)-benzamide

4-[3-(2-Chloro-phenyl)-5-ethoxy-3-methyl-2-oxo-2,3-dihydro-indole-1-sulphonyl]-N-(1,1-dimethyl-propyl)-benzamide is obtained in a similar way to Example 2, from laevorotatory 3-(2-chloro-phenyl)-5-ethoxy-3-methyl-1,3-dihydro-indol-2-one (Preparation 1) and sulphonyl chloride prepared in stage A of Example 4.

m.p.=138° C.

¹H NMR 400 MHz (DMSO-d₆): 0.82 (t, 3H); 1.24 (t, 3H); 1.32 (s, 6H); 1.71 (s, 3H); 1.78 (q, 2H); 3.85-3.92 (m, 2H); 6.37 (s, 1H); 6.93-6.96 (m, 1H); 7.22-7.28 (m, 1H); 7.34-7.38 (m, 1H); 7.42-7.48 (m, 1H); 7.75-7.81 (m, 2H); 7.98-8.01 (m, 2H); 8.08-8.11 (m, 2H)

### Example 5

### N-tert-Butyl-4-[3-(2-chloro-phenyl)-5-ethoxy-3-methyl-2-oxo-2,3-dihydro-indole-1-sulphonyl]-3-methoxybenzamide

N-tert-Butyl-4-[3-(2-chloro-phenyl)-5-ethoxy-3-methyl-2-oxo-2,3-dihydro-indole-1-sulphonyl]-3-methoxybenzamide is obtained in a similar way to Example 2, from laevorotatory 3-(2-chloro-phenyl)-5-ethoxy-3-methyl-1,3-dihydro-indol-2-one (Preparation

1) and 4-tert-butylcarbamoyl-2-methoxybenzenesulphonyl chloride described in document WO97/1556 (Preparation 13, Reagent (2).2).

m.p.=139° C.

¹H NMR 400 MHz (DMSO-d₆): 1.25 (t, 3H); 1.39 (s, 9H); 1.69 (s, 3H); 3.7 (s, 3H); 3.85-3.96 (m, 2H); 6.91-6.98 (m, 1H); 7.28-7.54 (m, 5H); 7.69-7.71 (m, 1H); 7.74-7.8 (m, 1H); 8-8.03 (m, 2H)

### Example 6

### N-tert-Butyl-4-[3-(2-fluoro-phenyl)-5-ethoxy-3-methyl-2-oxo-2,3-dihydro-indole-1-sulphonyl]-benzamide

N-tert-Butyl-4-[3-(2-fluoro-phenyl)-5-ethoxy-3-methyl-2-oxo-2,3-dihydro-indole-1-sulphonyl]-benzamide is obtained in a similar way to Example 5 from laevorotatory 3-(2-fluoro-phenyl)-5-ethoxy-3-methyl-1,3-dihydro-indol-2-one (Preparation 5).

m.p.=116° C.

¹H NMR 400 MHz (DMSO-d₆): 1.24 (t, 3H); 1.38 (s, 9H); 1.66 (s, 3H); 3.67 (s, 3H); 3.88-3.95 (m, 2H); 6.53 (s, 1H); 6.91-6.95 (m, 1H); 7.02-7.09 (m, 1H); 7.28-7.42 (m, 2H); 7.49-7.57 (m, 2H); 7.63-7.71 (m, 2H); 8-8.04 (m, 1H)

### Example 7

### 1-(4-Amino-2-methoxy-benzenesulphonyl)-3-(2-chloro-phenyl)-5-ethoxy-3-methyl-1,3-dihydro-indol-2-one

### A) 3-(2-Chloro-phenyl)-5-ethoxy-1-(2-methoxy-4-nitro-benzenesulphonyl)-3-methyl-1,3-dihydro-indol-2-one

Add 0.245 g of potassium tert-butylate to a solution of 0.6 g of laevorotatory 3-(2-chloro-phenyl)-5-ethoxy-3-methyl-1,3-dihydro-indol-2-one (Preparation 1) in 15 ml of tetrahydrofuran cooled to −30° C. Allow the temperature to return to 0° C. then cool to −60° C. and add 0.550 g of 4-nitro-2-methoxybenzenesulphonyl chloride. After stirring overnight at 20° C., treat with water and extract with ethyl acetate. Dry the organic phase over sodium sulphate, and evaporate to dryness to give the desired product in the form of a yellow foam, which is used directly in the next stage.

### B) 1-(4-Amino-2-methoxy-benzenesulphonyl)-3-(2-chloro-phenyl)-5-ethoxy-3-methyl-1,3-dihydro-indol-2-one

Reflux, for 3 h, a mixture of 1.028 g of the preceding compound (obtained in Stage A of Example 7), 0.56 g of iron powder, 7 ml of methanol and 1 ml of acetic acid, then partially evaporate the solvents under vacuum. Take up the residue in an aqueous solution of sodium bicarbonate, extract with ethyl acetate, filter on talc and decant. Dry the organic phase over sodium sulphate, and evaporate to dryness. Take up the residue in isopropyl ether, filter and dry, to give the desired product in the form of a white powder.

m.p.=211° C.

¹H NMR 250 MHz (DMSO-d₆): 1.25 (t, 3H); 1.66 (s, 3H); 3.5 (s, 3H); 3.81-3.97 (m, 2H); 6.15-6.21 (m, 2H); 6.29-6.35 (m, 1H); 6.88-6.91 (m, 1H); 7.29-7.48 (m, 3H); 7.52-7.54 (m, 1H); 7.65-7.7 (m, 1H); 7.72-7.79 (m, 1H)

### Example 8

### 3-{-4-[3-(2-Chloro-phenyl)-5-ethoxy-3-methyl-2-oxo-2,3-dihydro-indole-1-sulphonyl]-3-methoxy-phenyl}-1,1-diethyl-urea

### A) {4-[3-(2-Chloro-phenyl)-5-ethoxy-3-methyl-2-oxo-2,3-dihydro-indole-1-sulphonyl]-3-methoxy-phenyl}-phenyl carbamate

Add 1.5 ml of 2N aqueous sodium hydroxide solution then slowly 0.48 ml of phenyl chlorocarbonate to a solution of 0.53 g of the compound of Example 7 in 18 ml of tetrahydrofuran cooled to 0° C. After stirring overnight at 20° C., treat with water and extract with ethyl acetate. Dry the organic phase over sodium sulphate, evaporate to dryness, and purify by silica chromatography, eluting with a cyclohexane/ethyl acetate mixture to give the desired product in the form of a white foam, which is used directly in the next stage.

### B) 3-{4-[3-(2-Chloro-phenyl)-5-ethoxy-3-methyl-2-oxo-2,3-dihydro-indole-1-sulphonyl]-3-methoxy-phenyl}-1,1-diethyl-urea

Add 0.22 ml of diethylamine to a solution of 0.25 g of the preceding compound (stage A of Example 8) in 6.8 ml of dichloromethane cooled to 0° C. After stirring for 24 h at 20° C., treat with water and extract with dichloromethane. Dry the organic phase over sodium sulphate, evaporate to dryness, and purify by silica chromatography, eluting with a cyclohexane/ethyl acetate mixture to give the desired product in the form of a white powder.

m.p.=137° C.

¹H NMR 400 MHz (DMSO-d₆): 1.1 (t, 6H); 1.27 (t, 3H); 1.69 (s, 3H); 3.38-3.41 (m, 4H); 3.57 (s, 3H); 3.82-3.97 (m, 2H); 6.31 (m, 1H); 6.89-6.92 (m, 1H); 7.29-7.54 (m, 5H); 7.68-7.71 (m, 1H); 7.73-7.81 (m, 2H)

### Example 9

### 3-[1-(4-tert-Butylcarbamoyl-benzenesulphonyl)-5-ethoxy-3-methyl-2-oxo-2,3-dihydro-1H-indol-3-yl]-4-chloro-methyl benzoate

3-[1-(4-tert-Butylcarbamoyl-benzenesulphonyl)-5-ethoxy-3-methyl-2-oxo-2,3-dihydro-1H-indol-3-yl]-4-chloro-methyl benzoate is obtained in a similar way to Example 2, from laevorotatory 4-chloro-5-ethoxy-3-methyl-2-oxo-2,3-dihydro-1H-indol-3-yl)-methyl benzoate (Preparation 4-A).

m.p.=140° C.

¹H NMR 400 MHz (DMSO-d₆): 1.22 (t, 3H); 1.38 (s, 9H); 1.77 (s, 3H); 3.83-3.95 (m, 5H); 6.48 (m, 1H); 6.96-6.97 (m, 1H); 7.43-7.45 (m, 1H); 7.80-7.83 (m, 1H); 7.90-7.92 (m, 1H); 8.00-8.11 (m, 5H)

### Example 10

### 3-[1-(4-tert-Butylcarbamoyl-2-methoxy-benzenesulphonyl)-5-ethoxy-3-methyl-2-oxo-2,3-dihydro-1H-indol-3-yl]-4-chloro-methyl benzoate

3-[1-(4-tert-Butylcarbamoyl-2-methoxy-benzenesulphonyl)-5-ethoxy-3-methyl-2-oxo-2,3-dihydro-1H-indol-3-yl]-4-chloro-methyl benzoate is obtained in a similar way to Example 5 from laevorotatory 4-chloro-5-ethoxy-3-methyl-2-oxo-2,3-dihydro-1H-indol-3-yl)-methyl benzoate (Preparation 4-A).

m.p.=142° C.

¹H NMR 400 MHz (DMSO-d₆): 1.24 (t, 3H); 1.39 (s, 9H); 1.75 (s, 3H); 3.72 (s, 3H); 3.83-3.95 (m, 5H); 6.49 (m, 1H); 6.94-6.98 (m, 1H); 7.48-7.58 (m, 3H); 7.70-7.78 (m, 1H); 7.90-7.93 (m, 1H); 8.02-8.06 (m, 2H)

In the same conditions as for the preceding examples, the respective optical opposites of the preceding examples are obtained starting from dextrorotatory compounds III and appropriate sulphonyl chlorides.

In the following tables: Me=methyl and Et=ethyl.

Moreover, Z₂=H when Z₂=T₁W with T₁=—(CH₂)ₙ— with n=0 and W═H.

TABLE II

| | |
|---|---|
| | (I) |
| 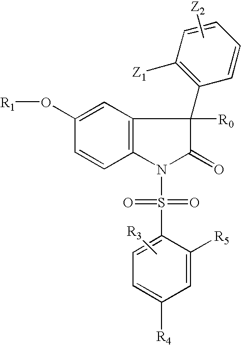 | |

| | | | | | | | | m.p. | |
|---|---|---|---|---|---|---|---|---|---|
| N° | R₀ | R₁ | Z₁ | Z₂ | R₄ | R₃ | R₅ | (° C.) | MH+ |
| 11 | Me | Et | Cl | H | OMe | H | OMe | 198 | 502 |
| 12 | Me | Et | Cl | H | 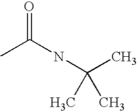 | H | H | 153 | 541 |
| 13 | Me | Et | Cl | H | 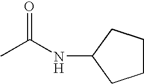 | H | H | 150 | 553 |
| 14 | Me | Et | Cl | H | 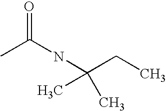 | H | H | 130 | 555 |
| 15 | Me | Et | Cl | H | 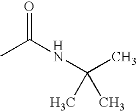 | H | OMe | 143 | 571 |
| 16 | Me | Et | F | H | 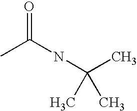 | H | OMe | 128 | 555 |
| 17 | Me | Et | Cl | H | —NH₂ | H | OMe | 119 | 487 |
| 18 | Me | Et | Cl | H | 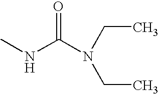 | H | OMe | 124 | 586 |

### Example 19

### 3-[1-(4-tert-Butylcarbamoyl-2-methoxy-benzenesulphonyl)-5-ethoxy-3-methyl-2-oxo-2,3-dihydro-1H-indol-3-yl]-4-chloro-benzoic acid

3-[1-(4-tert-Butylcarbamoyl-2-methoxy-benzenesulphonyl)-5-ethoxy-3-methyl-2-oxo-2,3-dihydro-1H-indol-3-yl]-4-chloro-benzoic acid is obtained in the same way as in Example 5 from laevorotatory 4-chloro-3-(5-ethoxy-3-methyl-2-oxo-2,3-dihydro-1H-indol-3-yl)-benzoic acid (Preparation 2)

m.p.=200° C.

¹H NMR 400 MHz (DMSO-d₆): 1.24 (t, 3H); 1.39 (s, 9H); 1.73 (s, 3H); 3.71 (s, 3H); 3.85-3.96 (m, 2H); 6.44-6.45 (m, 1H); 6.93-6.98 (m, 1H); 7.38-7.41 (m, 1H); 7.5-7.58 (m, 2H); 7.7-7.74 (m, 1H); 7.86-7.89 (m, 1H); 8.02-8.08 (m, 2H)

### Example 20

### N-tert-Butyl-4-{3-[2-chloro-5-(4-methyl-piperazine-1-carbonyl)-phenyl]-5-ethoxy-3-methyl-2-oxo-2,3-dihydro-indole-1-sulphonyl}-3-methoxy-benzamide

Add, in this order, 0.07 ml of N-methylpiperazine, 0.182 g of benzotriazolyl-N-oxytrisdimethylamino-phosphonium hexafluorophosphate and 0.1 ml of triethylamine to a solution of 0.2 g of 3-[1-(4-tert-butylcarbamoyl-2-methoxy-benzenesulphonyl)-5-ethoxy-3-methyl-2-oxo-2,3-dihydro-1H-indol-3-yl]-4-chloro-benzoic acid (Example 19) in 5 ml of dichloromethane cooled to 0° C. After stirring overnight at 20° C., add 5 ml of water, separate the organic phase on a hydrophobic cartridge, and purify by chromatography on a silica column, eluting with a dichloromethane/methanol mixture in the proportions 97/3 respectively, to obtain the expected product in the form of a white powder.

m.p.=96° C.

¹H NMR 400 MHz (DMSO-d₆): 1.25 (t, 3H); 1.39 (s, 9H); 1.70 (s, 3H); 2.22 (s, 3H); 2.31-2.48 (m, 4H); 3.25-3.35 (m, 4H); 3.72 (s, 3H); 3.95 (q, 2H); 6.92-6.99 (m, 1H); 7.38-7.4 (m, 2H); 7.5-7.59 (m, 2H); 7.7-7.73 (m, 2H); 8-8.05 (m, 2H)

In the same conditions we obtain the following examples starting from 3-[1-(4-tert-butylcarbamoyl-2-methoxy-benzenesulphonyl)-5-ethoxy-3-methyl-2-oxo-2,3-dihydro-1H-indol-3-yl]-4-chloro-benzoic acid (Example 19) and appropriate commercial amines.

TABLE III

| | |
|---|---|
| | (I) |
| 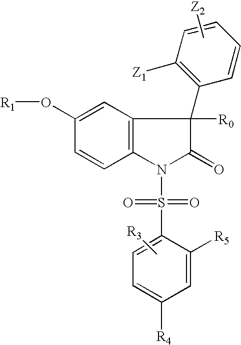 | |

| | | | | | | | | m.p. | |
|---|---|---|---|---|---|---|---|---|---|
| N° | R₀ | R₁ | Z₁ | Z₂ | R₄ | R₃ | R₅ | (° C.) | MH+ |
| 21 | Me | Et | Cl | 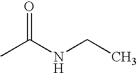 | 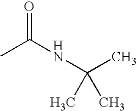 | H | OMe | 176 | 642 |
| 22 | Me | Et | Cl | 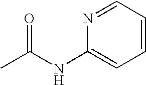 | 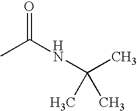 | H | OMe | 176 | 691 |
| 23 | Me | Et | Cl | 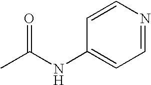 | 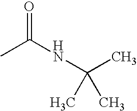 | H | OMe | 186 | 691 |
| 24 | Me | Et | Cl | 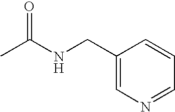 | 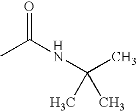 | H | OMe | 170 | 705 |
| 25 | Me | Et | Cl | 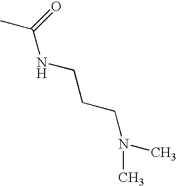 | 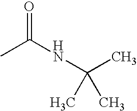 | H | OMe | 148 | 699 |
| 26 | Me | Et | Cl | 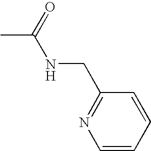 | 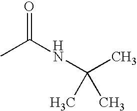 | H | OMe | 146 | 705 |
| 27 | Me | Et | Cl | 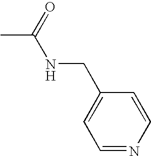 | 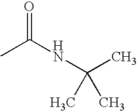 | H | OMe | 146 | 705 |
| 28 | Me | Et | Cl | 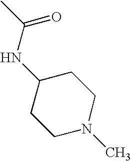 | 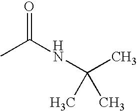 | H | OMe | 134 | 711 |
| 29 | Me | Et | Cl | 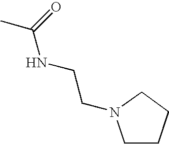 | 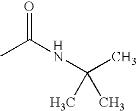 | H | OMe | 136 | 711 |
| 30 | Me | Et | Cl | 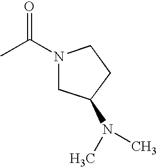 | 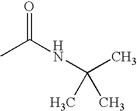 | H | OMe | 146 | 711 |
| 31 | Me | Et | Cl | 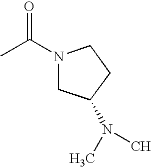 | 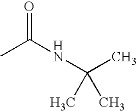 | H | OMe | 166 | 711 |
| 32 | Me | Et | Cl | 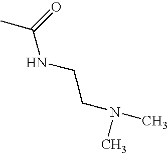 | 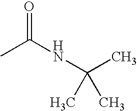 | H | OMe | 115 | 684 |
| 33 | Me | Et | Cl | 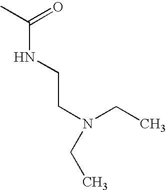 | 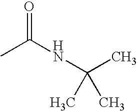 | H | OMe | 150 | 713 |
| 34 | Me | Et | Cl | 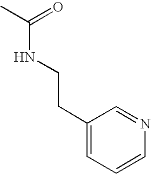 | 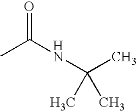 | H | OMe | 104 | 719 |
| 35 | Me | Et | Cl | 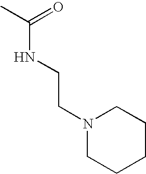 | 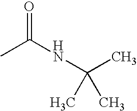 | H | OMe | 126 | 725 |
| 36 | Me | Et | Cl | 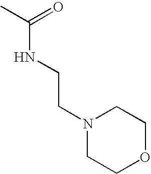 | 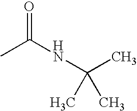 | H | OMe | 124 | 727 |
| 37 | Me | Et | Cl | 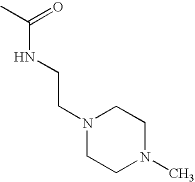 | 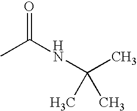 | H | OMe | 160 | 740 |
| 38 | Me | Et | Cl | 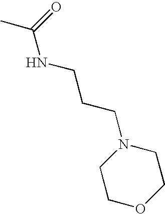 | 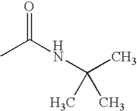 | H | OMe | 124 | 741 |
| 39 | Me | Et | Cl | 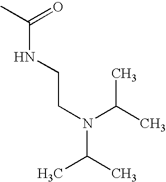 | 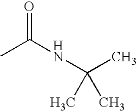 | H | OMe | 152 | 741 |
| 40 | Me | Et | Cl | 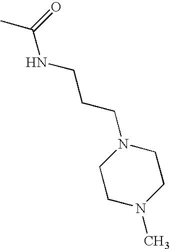 | 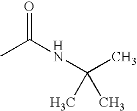 | H | OMe | 152 | 754 |
| 41 | Me | Et | Cl | 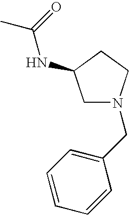 | 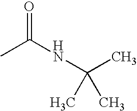 | H | OMe | 154 | 773 |
| 42 | Me | Et | Cl | 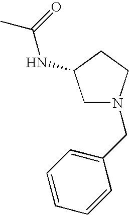 | 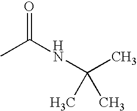 | H | OMe | 152 | 773 |
| 43 | Me | Et | Cl | 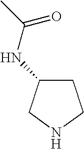 | 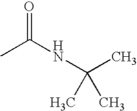 | H | OMe | 216 | 683 |
| 44 | Me | Et | Cl | 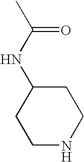 | 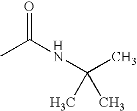 | H | OMe | 240 | 697 |

### Example 45

### N-tert-Butyl-4-{3-[2-chloro-5-(N′-ethyl-N′-(3-pyridyl)methylaminocarbonyl)-phenyl]-5-ethoxy-3-methyl-2-oxo-2,3-dihydro-indole-1-sulphonyl}-3-methoxy-benzamide

### A) Preparation of N-ethyl-3-pyridyl-methylamine

Add 5 g of 3-pyridylcarboxaldehyde to a mixture of 3.8 g of ethylamine hydrochloride, 60 ml of toluene, 110 ml of ethanol and 13.2 ml of triethylamine. After stirring for 5 minutes at 20° C., add 25 g of molecular sieve 4 A and continue stirring at 20° C. Filter off the insoluble matter, then wash with dichloromethane, evaporate to dryness and take up the residue in 50 ml of methanol. At a temperature of about 0° C., add 1.8 g of sodium borohydride then stir at 20° C. overnight. Evaporate the solvent under vacuum, take up the residue in dichloromethane, wash with 1N sodium hydroxide solution and then with an aqueous solution of sodium chloride, dry over sodium sulphate, evaporate the solvent and then distil under vacuum.

b.p.=77° C. at 530 Pa

¹H NMR 250 MHz (CDCl3): 1.18 (t, 3H); 2.73 (q, 4H); 3.85 (s, 2H); 7.25-7.32 (m, 1H); 7.65-7.75 (m, 1H); 8.50-8.65 (m, 2H)

### B) N-tert-Butyl-4-{3-[2-chloro-5-(N′-ethyl-N′-(3-pyridyl)methylamino-1-carbonyl)-phenyl]-5-ethoxy-3-methyl-2-oxo-2,3-dihydro-indole-1-sulphonyl}-3-methoxy-benzamide

In the conditions of Example 20, from 3-[1-(4-tert-Butylcarbamoyl-2-methoxy-benzenesulphonyl)-5-ethoxy-3-methyl-2-oxo-2,3-dihydro-1H-indol-3-yl]-4-chloro-benzoic acid (Example 19) and the preceding amine (stage A of Example 45), we obtain the desired compound in the form of a white powder.

m.p.=148° C.

MS[(+)ESI, m/z]: 733 (MH+)

### Example 46

### N-tert-Butyl-4-{3-[2-chloro-5-(1-methyl-hexahydro-pyrrolo[3,4-b]pyrrole-5-carbonyl)-phenyl]-5-ethoxy-3-methyl-2-oxo-2,3-dihydro-indole-1-sulphonyl}-3-methoxy-benzamide

In the conditions of Example 20, from 3-[1-(4-tert-butylcarbamoyl-2-methoxy-benzenesulphonyl)-5-ethoxy-3-methyl-2-oxo-2,3-dihydro-1H-indol-3-yl]-4-chloro-benzoic acid (Example 19) and 1-methyl-octahydro-pyrrolo[3,4-b]pyrrole obtained according to the conditions described in “Justus Liebigs Ann. Chemie 677, 154 (1964)”, we obtain the desired compound in the form of a white powder.

m.p.=130° C.

MS[(+)ESI, m/z]: 723 (MH+)

¹H NMR 400 MHz (DMSO-d₆): 1.23 (t, 3H); 1.38-1.41 (m, 9H); 1.42-1.61 (m, 1H); 1.71 (s, 3H); 1.82-2.08 (m, 1H); 2.11-2.21 (m, 3H); 2.23-2.3 (m, 1H); 2.7-2.82 (m, 1H); 2.95-3.05 (m, 1H); 3.33-3.71 (m, 4H); 3.72 (s, 3H); 3.87-4 (m, 2H); 6.4-6.46 (m, 1H); 6.92-7 (m, 1H); 7.32-7.4 (m, 1H); 7.45-7.58 (m, 3H); 7.68-7.72 (m, 1H); 7.79-7.88 (m, 1H); 7.99-8.09 (m, 2H)

### Example 47

### N-tert-Butyl-4-{3-[2-chloro-5-(2-aminoethylamino-carbonyl)-phenyl]-5-ethoxy-3-methyl-2-oxo-2,3-dihydro-indole-1-sulphonyl}-3-methoxy-benzamide

A) Add, in this order, 0.07 ml of 2-tert-butoxycarbonylamino-ethylamine, 0.139 g of benzotriazolyl-N-oxytrisdimethylamino phosphonium hexafluorophosphate and 0.08 ml of triethylamine to a solution of 0.15 g of 3-[1-(4-tert-butylcarbamoyl-2-methoxy-benzenesulphonyl)-5-ethoxy-3-methyl-2-oxo-2,3-dihydro-1H-indol-3-yl]-4-chloro-benzoic acid (Example 19) in 5 ml of dichloromethane cooled to 0° C. After stirring overnight at 20° C., add 5 ml of water, then separate the organic phase on a hydrophobic cartridge, purify by chromatography on a silica column, eluting with a dichloromethane/methanol mixture in the proportions 97/3 respectively, to obtain the expected product in the form of a white powder.

MS06/04/125: MS[(+), ESI, m/z]: 757 (MH+)

¹H NMR 400 MHz (DMSO-d₆): 1.22 (t, 3H); 1.35-1.42 (m, 18H); 1.76 (s, 3H); 3.1-3.19 (m, 2H); 3.29-3.32 (m, 2H); 3.72 (s, 3H); 3.82-3.97 (m, 2H); 6.42 (s, 1H); 6.9-6.99 (m, 2H); 7.5/7.58 (m, 2H); 7.7-7.75 (m, 1H); 7.78-7.81 (m, 1H); 8.02-8.09 (m, 2H)

B) The hydrochloride of the desired compound is obtained in the form of a white powder by deprotection of the preceding compound in an ethereal solution of hydrochloric acid.

m.p.=204° C.

MS[(+)ESI, m/z]: 657 (MH+)

### Example 48

### 3-[1-(4-tert-Butylcarbamoyl-benzenesulphonyl)-5-ethoxy-3-methyl-2-oxo-2,3-dihydro-1H-indol-3-yl]-4-chloro-benzoic acid

3-[1-(4-tert-Butylcarbamoyl-benzenesulphonyl)-5-ethoxy-3-methyl-2-oxo-2,3-dihydro-1H-indol-3-yl]-4-chloro-benzoic acid is obtained in a similar way to that described in Example 2, from laevorotatory 4-chloro-3-(5-ethoxy-3-methyl-2-oxo-2,3-dihydro-1H-indol-3-yl)-benzoic acid (Preparation 2).

m.p.=188° C.

¹H NMR 400 MHz (DMSO-d6): 1.23 (t, 3H); 1.38 (s, 9H); 1.77 (s, 3H); 3.84-3.95 (m, 2H); 6.47 (s, 1H); 6.92-6.98 (m, 1H); 7.37-7.4 (m, 1H); 7.78-7.9 (m, 2H); 7.98-8.04 (m, 2H); 8.08-8.11 (m, 3H)

### Example 49

### N-tert-Butyl-4-{3-[2-chloro-5-(3-pyridylmethylaminocarbonyl)phenyl]-5-ethoxy-3-methyl-2-oxo-2,3-dihydro-indole-1-sulphonyl}benzamide

N-tert-butyl-4-{3-[2-chloro-5-(3-pyridylmethylaminocarbonyl)phenyl]-5-ethoxy-3-methyl-2-oxo-2,3-dihydro-indole-1-sulphonyl}benzamide is obtained in the conditions of Example 20, from 3-[1-(4-tert-butylcarbamoyl-benzenesulphonyl)-5-ethoxy-3-methyl-2-oxo-2,3-dihydro-1H-indol-3-yl]-4-chloro-benzoic acid (Example 48) and 3-pyridylmethylamine.

m.p.=130° C.

¹H NMR 400 MHz (DMSO-d₆): 1.15-1.27 (m, 3H); 1.38 (s, 9H); 1.79 (s, 3H); 3.87-3.93 (m, 2H); 4.67 (d, 2H); 6.47 (s, 1H); 6.91-6.98 (m, 1H); 7.38-7.41 (m, 1H); 7.8-7.92 (m, 3H); 7.99-8.05 (m, 2H); 8.09-8.11 (m, 2H); 8.24 (s, 1H); 8.38-8.9 (m, 1H); 8.75-8.79 (m, 1H); 8.88-8.9 (m, 1H)

In the same conditions, the following examples are obtained starting from 3-[1-(4-tert-butylcarbamoyl-benzenesulphonyl)-5-ethoxy-3-methyl-2-oxo-2,3-dihydro-1H-indol-3-yl]-4-chloro-benzoic acid (Example 48) and appropriate commercial amines.

TABLE IV

| | |
|---|---|
| | (I) |
| 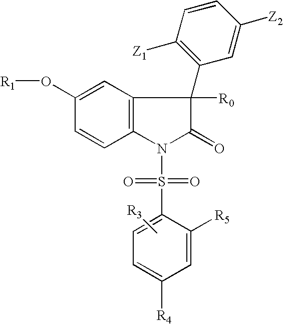 | |

| | | | | | | | | m.p. | |
|---|---|---|---|---|---|---|---|---|---|
| N° | R₀ | R₁ | Z₁ | Z₂ | R₄ | R₃ | R₅ | (° C.) | MH+ |
| 50 | Me | Et | Cl | 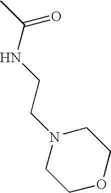 | 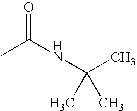 | H | H | 148 | 697 |
| 51 | Me | Et | Cl | 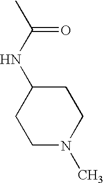 | 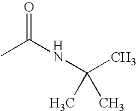 | H | H | 212 | 681 |
| 52 | Me | Et | Cl | 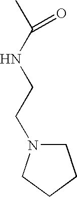 | 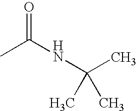 | H | H | 132 | 681 |
| 53 | Me | Et | Cl | 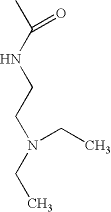 | 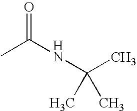 | H | H | 124 | 683 |

### Example 54

### N-Benzyl-4-chloro-3-{1-[4-(3,3-diethyl-ureido)-2-methoxy-benzenesulphonyl]-5-ethoxy-3-methyl-2-oxo-2,3-dihydro-1H-indol-3-yl}-benzamide

### A) 4-Chloro-3-[5-ethoxy-3-methyl-1-(4-nitro-benzenesulphonyl)-2-oxo-2,3-dihydro-1H-indol-3-yl]-benzoic acid

Add 0.340 g of potassium tert-butylate to a solution of 0.5 g of laevorotatory 4-chloro-3-(5-ethoxy-3-methyl-2-oxo-2,3-dihydro-1H-indol-3-yl)-benzoic acid (Preparation 2) in 7.5 ml of tetrahydrofuran cooled to −20° C. Allow the temperature to return to 0° C., cool to −20° C. and add 0.400 g of 4-nitro-2-methoxybenzenesulphonyl chloride.

After stirring overnight at 20° C., treat with water and extract with ethyl acetate.

Dry the organic phase over sodium sulphate, evaporate to dryness, purify by silica chromatography, eluting with a dichloromethane/methanol mixture in the proportions 93/7 respectively, to give the desired product in the form of a white powder.

¹H NMR 400 MHz (DMSO-d₆): 1.21 (t, 3H); 1.75 (s, 3H); 3.81 (s, 3H); 3.85-3.95 (m, 2H); 6.41-6.48 (m, 1H); 6.92-6.99 (m, 1H); 7.35-7.4 (m, 1H); 7.68-7.73 (m, 1H); 7.85-7.9 (m, 1H); 7.96-8.05 (m, 2H); 8.25-8.3 (m, 2H)

### B) 4-Chloro-3-[5-chloro-1-(2-methoxy-4-nitro-benzenesulphonyl)-3-methyl-2-oxo-2,3-dihydro-1H-indol-3-yl]-N-pyridin-3-ylmethyl-benzamide

Add, in this order, 0.12 ml of 3-pyridylmethylamine, 0.76 g of benzotriazolyl-N-oxytrisdimethylamino phosphonium hexafluorophosphate and 0.19 ml of triethylamine to a solution of 0.32 g of the acid prepared previously (stage A of Example 54) in 5 ml of dichloromethane cooled to 0° C. After stirring overnight at 20° C., add 5 ml of water, then separate the organic phase on a hydrophobic cartridge. Then purify by chromatography on a silica column, eluting with a dichloromethane/methanol mixture in the proportions 95/5 respectively, to obtain the expected product in the form of a yellow powder.

¹H NMR 250 MHz (DMSO-d₆): 1.2-1.3 (m, 3H); 1.79 (s, 3H); 3.81 (s, 3H); 3.85-3.99 (m, 2H); 4.54 (d. 2H); 6.48-6.5 (m, 1H); 6.95-7.01 (m, 1H); 7.35-7.48 (m, 2H); 7.69-7.8 (m, 2H); 7.85-7.9 (m, 1H); 7.99-8.06 (m, 2H); 8.2-8.33 (m, 2H); 8.46-8.5 (m, 1H); 8.59-8.62 (m, 1H)

### C) 3-[1-(4-Amino-2-methoxy-benzenesulphonyl)-5-chloro-3-methyl-2-oxo-2,3-dihydro-1H-indol-3-yl]-4-chloro-N-pyridin-3-ylmethyl-benzamide

Add 0.158 g of iron then 0.23 ml of acetic acid to 0.37 g of 4-chloro-3-[5-chloro-1-(2-methoxy-4-nitro-benzenesulphonyl)-3-methyl-2-oxo-2,3-dihydro-1H-indol-3-yl]-N-pyridin-3-ylmethyl-benzamide (stage B of Example 54) suspended in 5 ml of ethanol. Heat the reaction mixture with stirring under reflux for 3 hours. Partially evaporate the solvents at reduced pressure, then add 10 ml of a saturated aqueous solution of sodium bicarbonate and 10 ml of ethyl acetate. Stir the reaction mixture for 15 minutes at room temperature, then filter and rinse with ethyl acetate. After decanting, wash the organic phase with a saturated aqueous solution of sodium chloride. Dry the organic phase over Na₂SO₄, evaporate to dryness, and purify by silica chromatography, eluting with a dichloromethane/methanol mixture in the proportions 93/6 respectively, to give the desired product in the form of a white powder.

¹H NMR 250 MHz (DMSO-d₆): 1.21 (t, 3H); 1.73 (s, 3H); 3.51 (s, 3H); 3.8-3.95 (m, 2H); 4.55 (d, 2H); 6.15-6.24 (m, 2H); 6.39-6.42 (m, 1H); 6.88-6.95 (m, 1H); 7.35-7.48 (m, 2H); 7.56-7.61 (m, 1H); 7.65-7.7 (m, 1H); 7.75-7.89 (m, 2H); 8.19-8.21 (m, 1H); 8.47-8.5 (m, 1H); 8.58-8.62 (m, 1H)

### D) [4-(5-Chloro-3-{2-chloro-5-[(pyridin-3-ylmethyl)-carbamoyl]-phenyl}-3-methyl-2-oxo-2,3-dihydro-indole-1-sulphonyl)-3-methoxy-phenyl]-carbamic acid phenyl ester

Add 0.7 ml of a 1.5N aqueous sodium hydroxide solution and then slowly 0.08 ml of phenyl chlorocarbonate to a solution of 0.53 g of 3-[1-(4-amino-2-methoxy-benzenesulphonyl)-5-chloro-3-methyl-2-oxo-2,3-dihydro-1H-indol-3-yl]-4-chloro-N-pyridin-3-ylmethyl-benzamide (stage C of Example 54) in 13 ml of tetrahydrofuran cooled to 0° C. After stirring overnight at 20° C., treat with water and extract with ethyl acetate. Dry the organic phase over sodium sulphate, evaporate to dryness, purify by silica chromatography, eluting with a dichloromethane/methanol mixture 9/1, to give the desired product in the form of a white foam.

¹H NMR 400 MHz (DMSO-d₆): 1.22 (t, 3H); 1.69-1.8 (m, 3H); 3.59 (s, 3H); 3.82-3.95 (m, 2H); 4.49-4.58 (m, 2H); 6.4-6.45 (m, 1H); 6.72-6.8 (m, 1H); 6.88-6.98 (m, 1H); 7.12-7.3 (m, 3H); 7.35-7.48 (m, 4H); 7.65-7.75 (m, 2H); 7.81-7.98 (m, 2H); 8.15-8.21 (m, 1H); 8.41-8.49 (m, 1H); 8.56-8.61 (m, 1H); 9.25-9.32 (m, 1H)

### E) 4-Chloro-3-{5-chloro-1-[4-(3,3-diethyl-ureido)-2-methoxy-benzenesulphonyl]-3-methyl-2-oxo-2,3-dihydro-1H-indol-3-yl}-N-pyridin-3-ylmethyl-benzamide

Add 0.05 ml of diethylamine to a solution of 0.075 g of [4-(5-chloro-3-{2-chloro-5-[(pyridin-3-ylmethyl)-carbamoyl]-phenyl}-3-methyl-2-oxo-2,3-dihydro-indole-1-sulphonyl)-3-methoxy-phenyl]-carbamic acid phenyl ester (stage D of Example 54) in 1.5 ml of dichloromethane cooled to 0° C. After stirring for 24 h at 20° C., treat with water and separate the organic phase on a hydrophobic cartridge. Dry the organic phase over sodium sulphate, evaporate to dryness, and purify by silica chromatography, eluting with a dichloromethane/methanol mixture in the proportions 95/5 respectively, to give the desired product in the form of a white powder.

m.p.=148° C.

¹H NMR 400 MHz (DMSO-d₆): 1.09 (t, 6H); 1.24 (t, 3H); 1.74 (s, 3H); 3.32-3.4 (m, 4H); 3.58 (s, 3H); 3.82-3.91 (m, 2H); 4.5-4.55 (m, 2H); 6.42-6.43 (m, 1H); 6.89-6.93 (m, 1H); 7.29-7.45 (m, 3H); 7.52-7.55 (m, 1H); 7.68-7.88 (m, 4H); 8.18-8.21 (m, 1H); 8.45-8.5 (m, 1H); 8.57-8.6 (m, 1H)

### Example 55

### 4-[3-(5-Amino-2-chloro-phenyl)-5-ethoxy-3-methyl-2-oxo-2,3-dihydro-indole-1-sulphonyl]-N-tert-butyl-3-methoxy-benzamide

4-[3-(5-Amino-2-chloro-phenyl)-5-ethoxy-3-methyl-2-oxo-2,3-dihydro-indole-1-sulphonyl]-N-tert-butyl-3-methoxy-benzamide is obtained in the same way as in Example 5, from laevorotatory 3-(5-amino-2-chloro-phenyl)-5-ethoxy-3-methyl-2-oxo-2,3-dihydro-indole (Preparation 3).

m.p.=166° C.

¹H NMR 400 MHz (DMSO-d₆): 1.25 (t, 3H); 1.38 (s, 9H); 1.57 (s, 3H); 3.68 (s, 3H); 3.88-3.98 (m, 2H); 6.38 (m, 1H); 6.45-6.5 (m, 1H); 6.83-6.88 (m, 1H); 6.9-6.95 (m, 2H); 7.5-7.55 (m, 1H); 7.67-7.69 (m, 1H); 8-8.09 (m, 2H)

### Example 56

### N-{3-[1-(4-tert-butylcarbamoyl-2-methoxy-benzenesulphonyl)-5-ethoxy-3-methyl-2-oxo-2,3-dihydro-1H-indol-3-yl]-4-chloro-phenyl}nicotinamide

Slowly add 0.22 ml of triethylamine and 0.091 g of nicotinyl chloride hydrochloride to a solution of 0.15 g of the compound of Example 55 in 5 ml of dichloromethane cooled to 0° C. After stirring overnight at 20° C., hydrolyse with 5 ml of dilute ammonium chloride. Decant the organic phase, wash with water, and evaporate to dryness. Purify the residue by chromatography on a silica column, eluting with a dichloromethane/methanol gradient to obtain the expected product in the form of a white powder.

m.p.=192° C.

¹H NMR 400 MHz (DMSO-d₆): 1.25 (t, 3H); 1.39 (s, 9H); 1.69 (s, 3H); 3.71 (s, 3H); 3.88-3.98 (m, 2H); 6.92-6.98 (m, 1H); 7.3-7.32 (m, 1H); 7.5-7.65 (m, 3H); 7.7-7.77 (m, 1H); 7.85-7.88 (1H); 8.02-8.1 (m, 2H); 8.21 (m, 1H); 8.3-8.35 (m, 1H); 8.78-8.8 (m, 1H); 9.15 (m, 1H)

### Example 57

### N-tert-butyl-4-{3-[2-chloro-5-(2-dimethylamino-acetylamino)-phenyl]-5-ethoxy-3-methyl-2-oxo-2,3-dihydro-indole-1-sulphonyl}-3-methoxy-benzamide

Add, in this order, 0.053 g of N,N-dimethyl-glycine, 0.283 g of bis(2-oxo-3-oxazolidinyl)phosphinic chloride and 0.22 ml of triethylamine to a solution of 0.15 g of the compound from Example 55 in 5 ml of dichloromethane cooled to 0° C. After stirring overnight at 20° C., add 5 ml of water, separate the organic phase and wash it with water twice more, then evaporate under vacuum. Purify the residue by chromatography on a silica column, eluting with a dichloromethane/methanol gradient to obtain the expected product in the form of a white powder.

m.p.=150° C.

¹H NMR 400 MHz (DMSO-d₆): 1.25 (t, 3H); 1.38 (s, 9H); 1.65 (s, 3H); 2.29 (s, 6H); 3.09 (s, 2H); 3.7 (s, 3H); 3.88-3.98 (m, 2H); 6.9-6.96 (m, 1H); 7.19-7.22 (m, 1H); 7.48-7.55 (m, 2H); 7.68-7.71 (m, 1H); 7.75-7.81 (m, 1H); 8-8.08 (m, 3H)

In the same conditions, the following examples are obtained starting from 4-[3-(5-amino-2-chloro-phenyl)-5-ethoxy-3-methyl-2-oxo-2,3-dihydro-indole-1-sulphonyl]-N-tert-butyl-3-methoxy-benzamide (Example 55) and appropriate commercially available acids.

TABLE V

| | |
|---|---|
| | (I) |
| 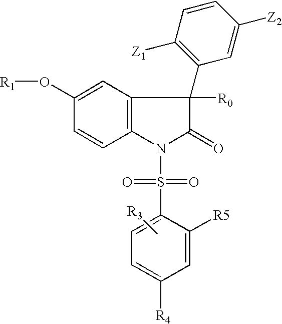 | |

| | | | | | | | | m.p. | |
|---|---|---|---|---|---|---|---|---|---|
| N° | R₀ | R₁ | Z₁ | Z₂ | R₄ | R₃ | R₅ | (° C.) | MH+ |
| 58 | Me | Et | Cl | 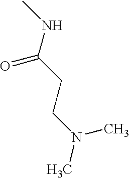 | 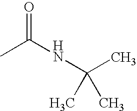 | H | OMe | 170 | 685 |
| 59 | Me | Et | Cl | 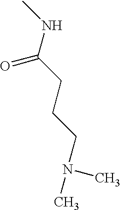 | 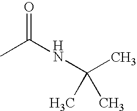 | H | OMe | 160 | 699 |
| 60 | Me | Et | Cl | 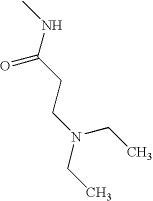 | 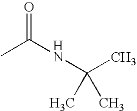 | H | OMe | 136 | 713 |
| 61 | Me | Et | Cl | 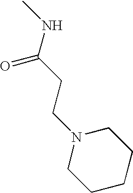 | 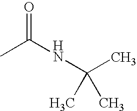 | H | OMe | 165 | 725 |
| 62 | Me | Et | Cl | 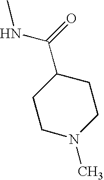 | 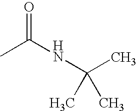 | H | OMe | 180 | 711 |

### Example 63

### N-tert-butyl-4-[3-(2-chloro-5-pyrrolidin-1-ylmethyl-phenyl)-5-ethoxy-3-methyl-2-oxo-2,3-dihydro-indole-1-sulphonyl]-3-methoxy-benzamide

### A) 3-(2-chloro-5-pyrrolidin-1-ylmethyl-phenyl)-5-ethoxy-3-methyl-2-oxo-2,3-dihydro-indole

Add, in this order, 0.19 ml of pyrrolidine, 0.243 g of sodium triacetoxyborohydride and 0.10 ml of acetic acid to a solution of 0.3 g of chiral 4-chloro-3-(5-ethoxy-3-methyl-2-oxo-2,3-dihydro-1H-indol-3-yl)-benzaldehyde (Preparation 4 C) in 6 ml of dichloromethane cooled to 0° C. After stirring overnight at 20° C., evaporate under vacuum, take up in 10 ml of ethyl acetate, wash with 10 ml of aqueous solution of sodium bicarbonate, separate the organic phase, dry it over sodium sulphate and then evaporate under vacuum. Purify the residue by chromatography on a silica column, eluting with a dichloromethane/methanol gradient to obtain the expected product in the form of a white powder.

MS[(+)ESI, m/z]: 385 (MH+)

¹H NMR 250 MHz (DMSO-d₆): 1.2 (t, 3H); 1.64 (s, 3H); 1.69-1.8 (m, 4H); 3.28-3.35 (m, 4H); 3.64-3.7 (m, 2H); 3.80-3.91 (m, 2H); 6.3-6.32 (m, 1H); 6.7-6.81 (m, 2H); 7.25-7.28 (m, 2H); 7.62-7.68 (m, 1H)

### B) N-tert-Butyl-4-[3-(2-chloro-5-pyrrolidin-1-ylmethyl-phenyl)-5-ethoxy-3-methyl-2-oxo-2,3-dihydro-indole-1-sulphonyl]-3-methoxy-benzamide

N-tert-butyl-4-[3-(2-chloro-5-pyrrolidin-1-ylmethyl-phenyl)-5-ethoxy-3-methyl-2-oxo-2,3-dihydro-indole-1-sulphonyl]-3-methoxy-benzamide is obtained in the same way as in Example 5, from the compound prepared previously (stage A of Example 63).

m.p.=132° C.

¹H NMR 400 MHz (DMSO-d₆): 1.22 (t, 3H); 1.39 (s, 9H); 1.69-1.79 (m, 7H); 2.4-2.52 (m, 4H); 3.6-3.71 (m, 5H); 3.85-3.95 (m, 2H); 6.92-6.98 (m, 1H); 7.21-7.31 (m, 2H); 7.49-7.58 (m, 2H); 7.65-7.71 (m, 2H); 8-8.06 (m, 2H)

In the same conditions, the following examples are obtained starting from chiral 4-chloro-3-(5-ethoxy-3-methyl-2-oxo-2,3-dihydro-1H-indol-3-yl)-benzaldehyde (Preparation 4 C) and appropriate commercial amines.

TABLE VI

| | |
|---|---|
| | (I) |
| 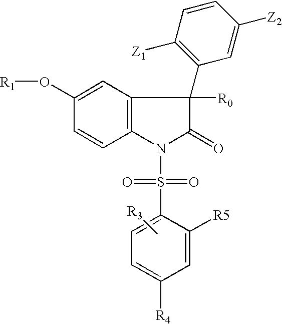 | |

| | | | | | | | | | Mass |
|---|---|---|---|---|---|---|---|---|---|
| | | | | | | | | m.p. | spectrometry: |
| N° | R₀ | R₁ | Z₁ | Z₂ | R₄ | R₃ | R₅ | (° C.) | MH+ |
| 64 | Me | Et | Cl | 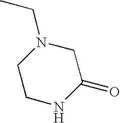 | 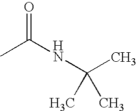 | H | OMe | 164 | 683 |
| 65 | Me | Et | Cl | 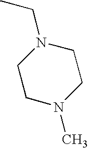 | 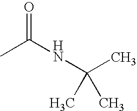 | H | OMe | 136 | 683 |

### Example 66

### N-(2-Fluoro-1,1-dimethylethyl)-4-[3-(2-chlorophenyl)-5-ethoxy-3-methyl-2-oxo-2,3-dihydroindole-1-sulphonyl]benzamide

### A) 4-[3-(2-Chlorophenyl)-5-ethoxy-3-methyl-2-oxo-2,3-dihydroindole-1-sulphonyl]benzoic acid

Add 0.70 g of a dispersion of sodium hydride at 60% in oil to the solution of 2 g of dextro-rotatory 3-(2-chlorophenyl)-5-ethoxy-3-methyl-1,3-dihydroindol-2-one (Preparation 1) in 20 ml of tetrahydrofuran cooled to −10° C. Allow the temperature to return to 0° C., cool to −10° C., then add 1.61 g of 4-chlorosulphonylbenzoic acid. After stirring at 20° C. for 15 hours, hydrolyse with water and extract with ethyl acetate. Dry the organic phase over sodium sulphate and evaporate to dryness. Purify the residue by silica chromatography, eluting with the gradient of the mixture DCM/MeOH of from (100/0; v/v) to (90/10; v/v), to give the desired product in the form of a white resin, m.p.=174° C.

### B) N-(2-Fluoro-1,1-dimethylethyl)-4-[3-(2-chlorophenyl)-5-ethoxy-3-methyl-2-oxo-2,3-dihydroindole-1-sulphonyl]benzamide

Add, in this order, 0.115 g of bis(2-oxo-3-oxazolidinyl)phosphinic chloride, 0.20 ml of triethylamine, then 0.063 g of 2-fluoro-1,1-dimethylethylamine prepared according to “Journal of Medicinal Chemistry, 1991, vol. 34, 29-37” to the solution of 0.2 g of 4-[3-(2-chlorophenyl)-5-ethoxy-3-methyl-2-oxo-2,3-dihydroindole-1-sulphonyl]benzoic acid prepared in A in 3 ml of dichloromethane cooled to 0° C. After stirring overnight at 20° C., add water, extract with dichloromethane, separate the organic phase on a hydrophobic cartridge, and purify by chromatography on a silica column, eluting with the gradient of the mixture DCM/MeOH of from (100/0; v/v) to (98/2; v/v), to give the desired product in the form of a white powder, m.p.=182° C.

¹H NMR 400 MHz (DMSO-d₆): 1.22 (t, 3H); 1.36 (s, 6H); 1.71 (s, 3H); 3.84-3.93 (m, 2H); 4.53 (s, 1H); 4.65 (s, 1H); 6.36 (s, 1H); 6.92-6.97 (m, 1H); 7.22-7.79 (m, 3H); 7.76-7.80 (m, 2H); 8.00-8.13 (m, 4H)

### Example 67

### tert-Butyl (2-{4-[3-(2-chlorophenyl)-5-ethoxy-3-methyl-2-oxo-2,3-dihydroindole-1-sulphonyl]benzoylamino}-2-methylpropyl)carbamate

### A) tert-Butyl (2-amino-2-methylpropyl)carbamate

Add 1.24 g of tert-butylcarbonic acid anhydride to a solution of 1 g of commercially available 2-methylpropane-1,2-diamine in 10 ml of acetonitrile cooled to 0° C. After stirring for 2 h at 0° C., allow the temperature to return to 20° C. for 1 h. Filter on a frit, evaporate the filtrate and purify the evaporation residue by chromatography on a silica column, eluting with the gradient of the mixture DCM/MeOH of from (100/0; v/v) to (85/15; v/v), to give the desired product in the form of crystals, m.p.=70° C.

### B) tert-Butyl (2-{4-[3-(2-chlorophenyl)-5-ethoxy-3-methyl-2-oxo-2,3-dihydro-indole-1-sulphonyl]benzoylamino}-2-methylpropyl)carbamate

Prepared under conditions similar to stage B of Example 66 using the same 4-[3-(2-chlorophenyl)-5-ethoxy-3-methyl-2-oxo-2,3-dihydroindole-1-sulphonypenzoic acid and tert-butyl (2-amino-2-methylpropyl)carbamate prepared in A.

¹H NMR 400 MHz (DMSO-d₆): 1.20 (t, 3H); 1.29 (s, 6H); 1.34 (s, 9H); 3.17-3.20 (m, 2H); 3.83-3.91 (m, 2H); 6.33 (s, 1H); 6.90-6.95 (m, 1H); 7.20-7.46 (m, 3H); 7.72-7.80 (m, 2H); 8.00-8.07 (m, 4H)

### Example 68

### N-(2-Amino-1,1-dimethylethyl)-4-[3-(2-chlorophenyl)-5-ethoxy-3-methyl-2-oxo-2,3-dihydroindole-1-sulphonyl]benzamide

The hydrochloride of the desired compound is obtained in the form of a white powder by deprotection of the compound of Example 67 in an ethereal solution of hydrochloric acid.

m.p.=172° C.

Under conditions similar to stage B of Example 66, the examples given in Table VII below are obtained starting from 4-[3-(2-chlorophenyl)-5-ethoxy-3-methyl-2-oxo-2,3-dihydro-indole-1-sulphonyl]benzoic acid prepared in A of Example 66 and appropriate commercially available amines.

TABLE VII

| | |
|---|---|
| | (I) |
| 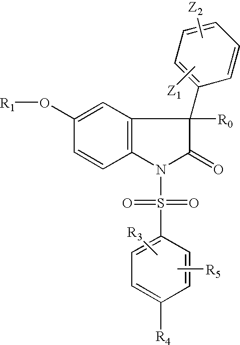 | |

| | | | | | | | | m.p. | |
|---|---|---|---|---|---|---|---|---|---|
| N° | R₀ | R₁ | Z₁ | Z₂ | R₄ | R₃ | R₅ | (° C.) | MH+ |
| 69 | Me | Et | 2-Cl | H | 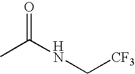 | H | H | 115 | 567 |
| 70 | Me | Et | 2-Cl | H | 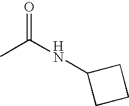 | H | H | 136 | 539 |
| 71 | Me | Et | 2-Cl | H | 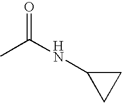 | H | H | 252 | 525 |
| 72 | Me | Et | 2-Cl | H | 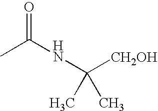 | H | H | 152 | 557 |
| 73 | Me | Et | 2-Cl | H | 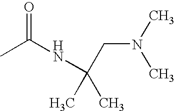 | H | H | 89 | 584 |
| 74 | Me | Et | 2-Cl | H | 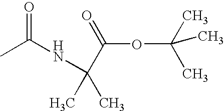 | H | H | 189 | 627 |

### Example 75

### N-tert-Butyl-4-{3-[2-chloro-5-(1-(2,2,2-trifluoroethyl)piperidin-4-ylamine-1-carbonyl)-phenyl]-5-ethoxy-3-methyl-2-oxo-2,3-dihydroindole-1-sulphonyl}benzamide

### A) 2,5-Dioxopyrrolidin-1-yl 3-[1-(4-tert-butylcarbamoylbenzenesulphonyl)-5-ethoxy-3-methyl-2-oxo-2,3-dihydro-1H-indol-3-yl]-4-chlorobenzoate

Add 0.61 g of N-hydroxysuccinimide to a solution of 3 g of 3-[1-(4-tert-butyl-carbamoylbenzenesulphonyl)-5-ethoxy-3-methyl-2-oxo-2,3-dihydro-1H-indol-3-yl]-4-chlorobenzoic acid (Example 48) in 34 ml of tetrahydrofuran, cooled to 0° C. After stirring for 20 min at 0° C., add 1.06 g of dicyclohexylcarbodiimide. After stirring overnight, evaporate the solvent under vacuum, take up the residue with dichloromethane, filter on a frit, evaporate the filtrate then purify the residue by chromatography on a silica column, eluting with the gradient of the mixture DCM/MeOH of from (100/0; v/v) to (95/5; v/v), to give the desired product in the form of a white powder, m.p.=168° C.

### B) N-tert-Butyl-4-{(3-[2-chloro-5-(1-(2,2,2-trifluoroethyl)piperidin-4-ylamine-1-carbon-yl)phenyl]-5-ethoxy-3-methyl-2-oxo-2,3-dihydroindole-1-sulphonyl}benzamide

Add 0.132 g of 1-(2,2,2-trifluoroethyl)piperidin-4-ylamine prepared according to WO 01/29042, p. 100, and 0.12 ml of triethylamine to a solution of 0.2 g of 2,5-dioxopyrrolidin-1-yl 3-[1-(4-tert-butylcarbamoylbenzenesulphonyl)-5-ethoxy-3-methyl-2-oxo-2,3-dihydro-1H-indol-3-yl]-4-chlorobenzoate prepared in A in 2.5 ml of tetrahydrofuran, cooled to 0° C. After stirring overnight at 20° C., add water, extract with dichloromethane, separate the organic phase on a hydrophobic cartridge, and purify by chromatography on a silica column, eluting with the gradient of the mixture DCM/MeOH of from (100/0; v/v) to (97/3; v/v), to give the desired product in the form of a white powder, m.p.=160° C.

¹H NMR 400 MHz (DMSO-d₆): 1.22 (t, 3H); 1.38 (s, 9H); 1.50-1.85 (m, 7H); 2.40-2.52 (m, 2H); 2.90-2.97 (m, 2H); 3.12-3.23 (m, 2H); 3.85-3.94 (m, 2H); 6.44-6.45 (m, 1H); 6.92-6.97 (m, 1H); 7.34-7.38 (m, 1H); 7.76-8.15 (m, 7H)

Under conditions similar to stage B of Example 75, the examples given in Table VIII below are obtained starting from 2,5-dioxopyrrolidin-1-yl 3-[1-(4-tert-butylcarbamoyl-benzenesulphonyl)-5-ethoxy-3-methyl-2-oxo-2,3-dihydro-1H-indol-3-yl]-4-chlorobenzoate prepared in A of Example 75 and appropriate commercially available amines or appropriate amines prepared according to the described processes.

TABLE VIII

| | |
|---|---|
| | (I) |
| 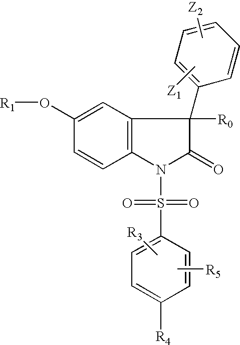 | |

| | | | | | | | | m.p. | |
|---|---|---|---|---|---|---|---|---|---|
| N° | R₀ | R₁ | Z₁ | Z₂ | R₄ | R₃ | R₅ | (° C.) | MH+ |
| 76 | Me | Et | 2-Cl | 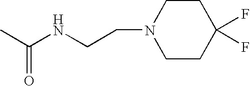 | 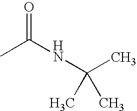 | H | H | 148 | 731 |
| 77 | Me | Et | 2-Cl | 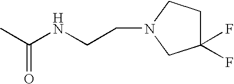 | 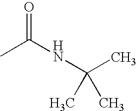 | H | H | 136 | 717 |
| 78 | Me | Et | 2-Cl | 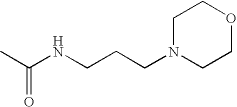 | 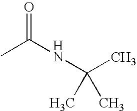 | H | H | 112 | 711 |
| 79 | Me | Et | 2-Cl | 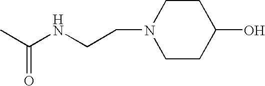 | 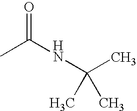 | H | H | 148 | 711 |
| 80 | Me | Et | 2-Cl | 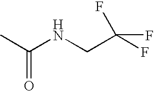 | 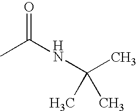 | H | H | 140 | 666 |
| 81 | Me | Et | 2-Cl | 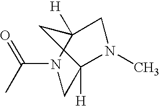 | 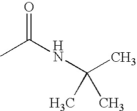 | H | H | 152 | 679 |
| 82 | Me | Et | 2-Cl | 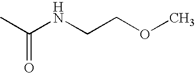 | 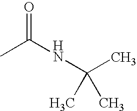 | H | H | 126 | 642 |
| 83 | Me | Et | 2-Cl | 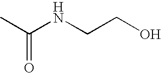 | 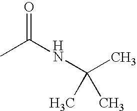 | H | H | 150 | 628 |
| 84 | Me | Et | 2-Cl | 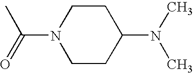 | 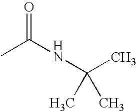 | H | H | 132 | 695 |
| 85 | Me | Et | 2-Cl | 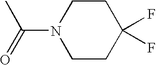 | 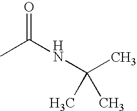 | H | H | 150 | 688 |
| 86 | Me | Et | 2-Cl | 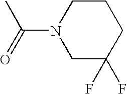 | 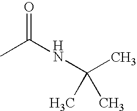 | H | H | 130 | 688 |
| 87 | Me | Et | 2-Cl | 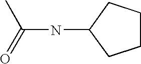 | 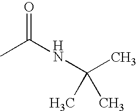 | H | H | 164 | 652 |
| 88 | Me | Et | 2-Cl | 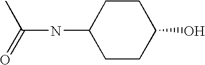 | 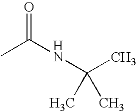 | H | H | 172 | 682 |
| 89 | Me | Et | 2-Cl | 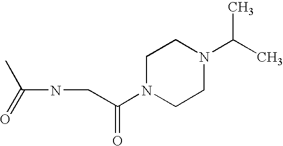 | 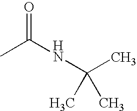 | H | H | 168 | 752 |
| 90 | Me | Et | 2-Cl | 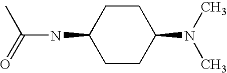 | 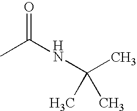 | H | H | 180 | 709 |
| 91 | Me | Et | 2-Cl | 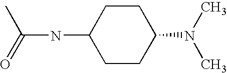 | 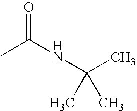 | H | H | 174 | 709 |

### Example 92

### (2-Dimethylamino)ethyl 3-[1-(4-tert-butylcarbamoylbenzenesulphonyl)-5-ethoxy-3-methyl-2-oxo-2,3-dihydro-1H-indol-3-yl]-4-chlorobenzoate

Add 0.116 g of bis(2-oxo-3-oxazolidinyl)phosphinic chloride, 0.07 ml of N,N-dimethyl-2-hydroxyethylamine and 0.14 ml of triethylamine to a solution of 0.2 g of 3-[1-(4-tert-butylcarbamoylbenzenesulphonyl)-5-ethoxy-3-methyl-2-oxo-2,3-dihydro-1H-indol-3-yl]-4-chlorobenzoic acid (Example 48) in 2.5 ml of dioxane, cooled to 0° C. After stirring overnight at 20° C., partially evaporate the solvent under vacuum, add 10 ml of an aqueous solution of NaHCO₃, extract with ethyl acetate, separate the organic phase and dry it over sodium sulphate, then evaporate under vacuum. Purify the residue by chromatography on a silica column, eluting with the gradient of the mixture DCM/MeOH of from (100/0; v/v) to (90/10; v/v), to give the desired product in the form of a white powder, m.p.=118° C.

¹H NMR 400 MHz (DMSO-d₆): 1.22 (t, 3H); 1.38 (s, 9H); 1.77 (s, 3H); 2.23 (s, 6H); 2.60-2.67 (m, 2H); 3.84-3.93 (m, 2H); 4.33-4.46 (m, 2H); 6.48-6.50 (m, 1H); 6.94-6.98 (m, 1H); 7.43-7.46 (m, 1H); 7.80-8.15 (m, 7H)

Under conditions similar to Example 92, the examples given in Table IX below are obtained starting from 3-[1-(4-tert-butylcarbamoylbenzenesulphonyl)-5-ethoxy-3-methyl-2-oxo-2,3-dihydro-1H-indol-3-yl]-4-chlorobenzoic acid (Example 48) and appropriate commercially available alcohols.

TABLE IX

| | |
|---|---|
| | (I) |
| 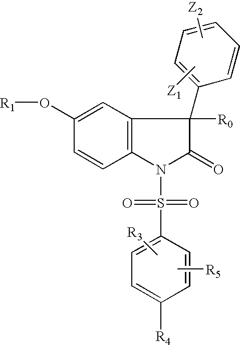 | |

| | | | | | | | | m.p. | |
|---|---|---|---|---|---|---|---|---|---|
| N° | R₀ | R₁ | Z₁ | Z₂ | R₄ | R₃ | R₅ | (° C.) | MH+ |
| 93 | Me | Et | 2-Cl | 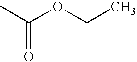 | 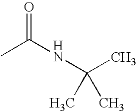 | H | H | 118 | 613 |
| 94 | Me | Et | 2-Cl | 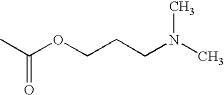 | 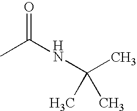 | H | H | 108 | 670 |
| 95 | Me | Et | 2-Cl | 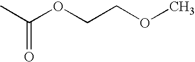 | 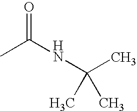 | H | H | 110 | 643 |
| 96 | Me | Et | 2-Cl | 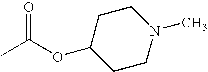 | 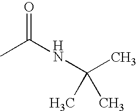 | H | H | 204 | 682 |

Under the sulphonylation conditions described in Example 2 and using compounds III, derived from preparations 5 to 19 above and appropriate benzenesulphonyl chlorides previously described, the compounds of the examples given in Table X below are obtained:

TABLE X

| | |
|---|---|
| | (I) |
| 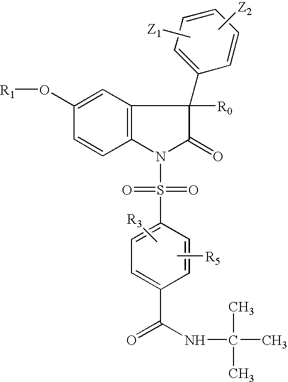 | |

| | No. of preparation | | | | | | | | |
|---|---|---|---|---|---|---|---|---|---|
| | of the precursor | | | | | | | m.p. | |
| N° | compound III | R₀ | R₁ | Z₁ | Z₂ | R₅ | R₃ | (° C.) | MH+ |
| 97 | 6 dextrorotatory | Me | Et | H | H | H | H | 83 | 507 |
| 98 | 6 laevorotatory | Me | Et | H | H | H | H | 89 | 507 |
| 99 | 6 dextrorotatory | Me | Et | H | H | H | 2-OMe | 148 | 537 |
| 100 | 6 laevorotatory | Me | Et | H | H | H | 2-OMe | 151 | 537 |
| 101 | 5 dextrorotatory | Me | Et | 2-F | H | H | H | 116 | 525 |
| 102 | 5 laevorotatory | Me | Et | 2-F | H | H | H | 114 | 525 |
| 103 | 7 dextrorotatory | Me | Et | 3-OMe | H | H | H | 101 | 537 |
| 104 | 7 laevorotatory | Me | Et | 3-OMe | H | H | H | 100 | 537 |
| 105 | 7 dextrorotatory | Me | Et | 3-OMe | H | H | 2-OMe | 108 | 567 |
| 106 | 8 dextrorotatory | Me | Et | 2-OMe | H | H | H | 98 | 537 |
| 107 | 8 laevorotatory | Me | Et | 2-OMe | H | H | H | 135 | 537 |
| 108 | 8 dextrorotatory | Me | Et | 2-OMe | H | H | 2-OMe | 146 | 567 |
| 109 | 8 laevorotatory | Me | Et | 2-OMe | H | H | 2-OMe | 140 | 567 |
| 110 | 9 dextrorotatory | Me | Et | 4-Me | H | H | H | 177 | 521 |
| 111 | 9 dextrorotatory | Me | Et | 4-Me | H | H | 2-OMe | 207 | 551 |
| 112 | 9 laevorotatory | Me | Et | 4-Me | H | H | 2-OMe | 208 | 551 |
| 113 | 10 dextrorotatory | Me | Et | 3-Me | H | H | H | 108 | 521 |
| 114 | 10 laevorotatory | Me | Et | 3-Me | H | H | H | 93 | 521 |
| 115 | 10 dextrorotatory | Me | Et | 3-Me | H | H | 2-OMe | 186 | 551 |
| 116 | 11 dextrorotatory | Me | Et | 2-OCF3 | H | H | H | 172 | 591 |
| 117 | 11 laevorotatory | Me | Et | 2-OCF3 | H | H | H | 177 | 591 |
| 118 | 11 dextrorotatory | Me | Et | 2-OCF3 | H | H | 2-OMe | 213 | 621 |
| 119 | 12 dextrorotatory | Me | Et | 2-CF3 | H | H | H | 144 | 575 |
| 120 | 12 laevorotatory | Me | Et | 2-CF3 | H | H | H | 144 | 575 |
| 121 | 12 dextrorotatory | Me | Et | 2-CF3 | H | H | 2-OMe | 214 | 605 |
| 122 | 13 dextrorotatory | Me | Et | 2-Cl | 3-Cl | H | H | 124 | 575 |
| 123 | 13 laevorotatory | Me | Et | 2-Cl | 3-Cl | H | H | 170 | 575 |
| 124 | 14 dextrorotatory | Me | Et | 2-Cl | 6-F | H | H | 188 | 559 |
| 125 | 14 laevorotatory | Me | Et | 2-Cl | 6-F | H | H | 128 | 559 |
| 126 | 14 dextrorotatory | Me | Et | 2-Cl | 6-F | H | 2-OMe | 133 | 589 |
| 127 | 15 dextrorotatory | Me | Et | 2-Cl | 5-OMe | H | H | 121 | 571 |
| 128 | 15 laevorotatory | Me | Et | 2-Cl | 5-OMe | H | H | 161 | 571 |
| 129 | 15 laevorotatory | Me | Et | 2-Cl | 5-OMe | H | 2-OMe | 128 | 601 |
| 130 | 16 dextrorotatory | Me | Et | 2-OBn | H | H | H | 109 | 613 |
| 131 | 16 dextrorotatory | Me | Et | 2-OBn | H | H | 2-OMe | 139 | 643 |
| 132 | 17 dextrorotatory | Me | Et | 4-OBn | H | H | H | 142 | 613 |
| 133 | 17 laevorotatory | Me | Et | 4-OBn | H | H | H | 143 | 163 |
| 134 | 17 dextrorotatory | Me | Et | 4-OBn | H | H | 2-OMe | 113 | 643 |
| 135 | 18 laevorotatory | Et | Et | H | H | H | H | 127 | 555 |
| 136 | 18 laevorotatory | Et | Et | H | H | H | 2-OMe | 139 | 585 |
| 137 | 19 laevorotatory | Pr | Et | H | H | H | H | 146 | 569 |

### Example 138

### N-tert-Butyl-4-[5-ethoxy-3-(2-hydroxyphenyl)-3-methyl-2-oxo-2,3-dihydroindole-1-sulphonyl]benzamide

Add 0.06 g of palladium-on-charcoal powder (10% by mass) and 0.19 g of ammonium formate to a solution of 0.3 g of the compound of Example 130 in 5 ml of methanol. After stirring for 8 hours under reflux, cool, filter on a bed of Celite and evaporate the solvent under vacuum. Take up the residue with dichloromethane, wash with an aqueous solution of sodium chloride, separate the organic phase and dry it over sodium sulphate, then evaporate under vacuum. Purify the residue by chromatography on a silica column, eluting with the gradient of the mixture cyclohexane/EtOAc of from (100/0; v/v) to (70/30; v/v), to give the desired product in the form of a white powder, m.p.=180° C.

¹H NMR 400 MHz (DMSO-d₆): 1.23 (t, 3H); 1.38 (s, 9H); 1.49 (s, 3H); 3.83-3.92 (m, 2H); 6.36-8.06 (m, 11H)

Under similar conditions, using the compounds of Examples 131, 132, 133 and 134, the compounds of the examples given in Table XI below are obtained:

TABLE XI

| | |
|---|---|
| | (I) |
| 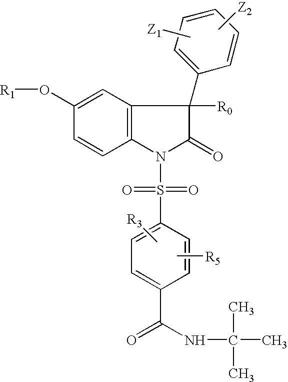 | |

| | Precursor | | | | | | | | |
|---|---|---|---|---|---|---|---|---|---|
| N° | Example | | | | | | | m.p. | |
| ex | No. | R₀ | R₁ | Z₁ | Z₂ | R₅ | R₃ | (° C.) | MH+ |
| 139 | 131 | Me | Et | 2-OH | H | H | 2-OMe | 203 | 553 |
| 140 | 132 | Me | Et | 4-OH | H | H | H | 137 | 523 |
| 141 | 133 | Me | Et | 4-OH | H | H | H | 134 | 523 |
| 142 | 134 | Me | Et | 4-OH | H | H | 2-OMe | 137 | 553 |

### Example 143

### N-tert-Butyl-4-[3-(2-chlorophenyl)-3-methyl-2-oxo-5-(2,2,2-trifluoroethoxy)-2,3-di-hydroindole-1-sulphonyl]benzamide

### A) N-tert-Butyl-4-[3-(2-chlorophenyl)-5-hydroxy-3-methyl-2-oxo-2,3-dihydroindole-1-sulphonyl]benzamide

Add 27.72 ml of a 1M solution of boron tribromide in DCM to a solution of 3 g of N-tert-butyl-4-[3-(2-chlorophenyl)-5-ethoxy-3-methyl-2-oxo-2,3-dihydroindole-1-sulphonyl]-benzamide (compound of Example 12) in solution in 75 ml of DCM, cooled to 0° C. Allow to stir at 20° C. for 16 hours. Run the medium onto ice and slowly add 45 ml of triethyl-amine. Wash the organic phase with water, dry over sodium sulphate and concentrate at reduced pressure. Purify the residue by chromatography on a silica column, eluting with the gradient of the mixture DCM/MeOH of from (100/0; v/v) to (97/3; v/v), to give the desired product in the form of a white powder, m.p.=262° C.

MH+=513

### B) N-tert-Butyl-4-[3-(2-chlorophenyl)-3-methyl-2-oxo-5-(2,2,2-trifluoroethoxy)-2,3-dihydroindole-1-sulphonyl]benzamide

Add 0.137 g of caesium carbonate, then 0.098 g of 2,2,2-trifluoroethyl trifluoromethane-sulphonate to a solution of 0.18 g of N-tert-butyl-4-[3-(2-chlorophenyl)-5-hydroxy-3-methyl-2-oxo-2,3-dihydroindole-1-sulphonyl]benzamide in solution in 3 ml of acetonitrile, cooled to 0° C. Allow to stir at 20° C. for 16 hours, then partially evaporate the solvent under vacuum. Take up the residue with dichloromethane and wash with an aqueous solution of sodium chloride. Dry the organic phase over sodium sulphate and concentrate at reduced pressure. Purify the residue by chromatography on a silica column, eluting with the gradient of the mixture cyclohexane/dichloromethane of from (30/70; v/v) to (0/100; v/v), to give the desired product in the form of a white powder, m.p.=134° C.

¹H NMR 400 MHz (DMSO-d₅): 1.39 (s, 9H); 1.72 (s, 3H); 4.62-4.70 (m, 2H); 6.57-6.59 (m, 1H); 7.08-7.5 (m, 4H); 7.75-8.12 (m, 6H)

MH+=595

### Example 144

### N-tert-Butyl-4-[3-(2-chlorophenyl)-3-methyl-2-oxo-5-hydroxy)-2,3-dihydroindole-1-sulphonyl]-3-methoxybenzamide

Obtained in a manner similar to stage A of Example 143, using the compound of Example 15.

m.p.=166° C.

NMR07/06/327 ¹H NMR 400 MHz (DMSO-d6): 1.38 (s, 9H); 1.65 (s, 3H); 3.69 (s, 3H); 6.16-6.17 (m, 1H); 6.73-6.77 (m, 1H); 7.29-8.05 (m, 8H)

MH+=543

### Example 145

### N-tert-Butyl-4-{3-[3-(3-dimethylaminopropoxy)phenyl]-5-ethoxy-3-methyl-2-oxo-2,3-dihydroindole-1-sulphonyl}benzamide

### A) N-tert-Butyl-4-[3-(3-hydroxyphenyl)-5-ethoxy-3-methyl-2-oxo-2,3-dihydroindole-1-sulphonyl]benzamide

Add 3.66 ml of a 1M solution of boron tribromide in DCM to a solution of 0.98 g of N-tert-butyl-4-[3-(3-methoxyphenyl)-5-ethoxy-3-methyl-2-oxo-2,3-dihydroindole-1-sulphonyl]benzamide (compound of Example 103) in solution in 30 ml of DCM, cooled to 0° C. Allow to stir at 0° C. for 30 min, then run the medium over ice and slowly add 1.5 ml of triethyl-amine. Wash the organic phase with water, dry over sodium sulphate and concentrate at reduced pressure. Purify the residue by chromatography on a silica column, eluting with the gradient of the mixture cyclohexane/EtOAc of from (100/0; v/v) to (70/30; v/v), to give the desired product in the form of a white powder, m.p.=196° C.

MH+=523

### B) N-tert-Butyl-4-{3-[3-(3-chloropropoxy)phenyl]-5-ethoxy-3-methyl-2-oxo-2,3-di-hydroindole-1-sulphonyl}benzamide TLC.ALF6.132

Add 0.15 g of caesium carbonate then 0.094 g of 1-chloro-3-iodopropane to a solution of 0.2 g of N-tert-butyl-4-[3-(3-hydroxyphenyl)-5-ethoxy-3-methyl-2-oxo-2,3-dihydroindole-1-sulphonyl]benzamide in solution in 4 ml of acetonitrile, cooled to 0° C. Allow to stir at 20° C. for 16 hours, then add an aqueous solution of sodium chloride. Dry the organic phase over sodium sulphate and concentrate at reduced pressure. Purify the residue by chromatography on a silica column, eluting with the gradient of the mixture cyclohexane/EtOAc of from (100/0; v/v) to (60/40; v/v), to give the desired product.

¹H NMR 400 MHz (DMSO-d₆): 1.27 (t, 3H); 1.37 (s, 9H); 1.64 (s, 3H); 2.08-2.16 (m, 2H); 3.75 (t, 2H); 3.94-4.02 (m, 4H); 6.50-8.05 (m, 11H)

MH+=599

### C) N-tert-Butyl-4-{3-[3-(3-dimethylaminopropoxy)phenyl]-5-ethoxy-3-methyl-2-oxo-2,3-dihydroindole-1-sulphonyl}benzamide SAR127943-1 ALF6.151

In an autoclave, heat at around 60° C., for 24 hours, the mixture of 0.07 g of N-tert-butyl-4-{3-[3-(3-chloropropoxy)phenyl]-5-ethoxy-3-methyl-2-oxo-2,3-dihydroindole-1-sulphonyl}-benzamide, 2.5 ml of DMF, 0.018 g of sodium iodide, 0.038 g of sodium carbonate and 1.2 ml of a 2M solution of dimethylamine in THF. Add water, extract with ethyl acetate, dry the organic phase, evaporate to dryness, and purify the residue by chromatography on a silica column, eluting with the gradient of the mixture DCM/MeOH of from (100/0; v/v) to (85/15; v/v), to give the desired product, m.p.=86° C.

¹H NMR 400 MHz (DMSO-d₆): 1.27 (t, 3H); 1.37 (s, 9H); 1.64 (s, 3H); 1.77-1.85 (m, 2H); 2.19 (s, 6H); 2.32-2.46 (m, 2H); 3.80-4.01 (m, 4H); 6.49-8.03 (m, 11H)

MH+=608

The compounds of the invention underwent pharmacological testing, which demonstrated their advantages as active substances in therapeutics.

Notably they were tested for their effects. More particularly, the affinity of the compounds of the invention for the V₂ vasopressin receptors was determined in an in vitro binding test, by the procedure described below.

In the following:
EDTA=ethylenediaminetetraacetic acid,
BSA=Bovine Serum Albumin,
AVP=vasopressin,
DMSO=dimethylsulphoxide.
   A. Measurement of affinity in vitro—IC₅₀:

The affinity of the compounds of the invention for the V₂ vasopressin receptors was measured in in vitro binding tests, as described in J. Pharmacol. Exp. Ther., (2002), 300: pp. 1122-1130.

Plasma membranes (about 20 μg/ml) obtained from CHO tissue or cell line expressing human recombinant V₂ vasopressin receptors, are incubated for 45 minutes at 25° C. in 200 μL of TRIS-HCl buffer (50 mM; pH 8.2) containing 2 mM of MgCl₂, 1 mM of EDTA, 0.1% of BSA, 1/500 protease inhibitor cocktail (Sigma #P2714) and 3.5 nM of [H3]-AVP. The reaction is stopped by filtration and washing on GF/B filters. Nonspecific binding is determined in the presence of 1 μM of AVP. The compounds of the invention, dissolved beforehand to a concentration of 10⁻² M in DMSO, are tested in a dilution series.

For each concentration the results are expressed as percentage inhibition of specific binding. An IC₅₀ (concentration of product inhibiting 50% of specific binding) is determined for each of the products using the in-house software Biost@t-SPEED v1.3 which employs the 4-parameter logistic model of Ratkovsky and Reedy (1986). Adjustment is obtained by nonlinear regression using Marquardt's algorithm of the SAS v8.2 software running under UNIX.

The affinity of the compounds of the invention for the Vᵢₐ and V_{1b} vasopressin receptors and the affinity of the compounds of the invention for the oxytocin (OXT) receptors were also tested.

The affinity of the compounds according to the invention for the OXT receptors was determined in an in vitro binding test using the method described by J. Elands et al. in Eur. J. Pharmacol. 1987, 147, 197-207. This method comprises in vitro investigation of the displacement of a radioiodated analogue of oxytocin from the oxytocin receptors in a membrane preparation of human oxytocin receptors.

The affinity of the compounds according to the invention for human Vᵢₐ receptors was determined according to the method described by M. Thibonnier et al. in J. Biol. Chem. 1994, 269, 3304-3310. The affinity of the compounds according to the invention for the V_{1b} receptors was determined according to the method described by T. Sugimoto et al. in J. Biol. Chem. 1994, 269, 27088-27092.

As shown in Table A below, the compounds of the present invention have high affinity and selectivity for the V₂ vasopressin receptors.

The compounds obtained according to examples of the present invention, shown in Table A below, are not limiting and are only for illustrating the invention.

TABLE A

| | | IC50 | | | | | |
|---|---|---|---|---|---|---|---|
| Ex. | IC50 V₂ | OXT | IC50 V₁ₐ | IC50 V_{1b} | IC50 | IC50 | IC50 |
| N^{o} | (nM) | (nM) | (nM) | (nM) | OXT/IC50 V₂ | V₁ₐ/IC50 V₂ | V_{1b}/IC50 V₂ |
| 12 | 1.9 | 140 | 450 | >1000 | 74 | >100 | >100 |
| 21 | 3.0 | >1000 | 340 | >1000 | >100 | >100 | >100 |
| 36 | 5.2 | 350 | >1000 | >1000 | 67 | >100 | >100 |
| 43 | 1.3 | 700 | 730 | >1000 | >100 | >100 | >100 |
| 62 | 2.5 | >1000 | 630 | >1000 | >100 | >100 | >100 |
| 72 | 1.3 | 390 | >1000 | 940 | >100 | >100 | >100 |
| 99 | 1.0 | 700 | 260 | >1000 | >100 | >100 | >100 |
| 144 | 2.6 | 130 | 310 | >1000 | 50 | >100 | >100 |

In order to illustrate the selectivity of the compounds according to the invention, the affinities measured on the V₁ₐ, V_{1b} and OXT receptors were compared with that measured on the V₂ receptor in Table A above. It is considered that when the IC₅₀ values are greater than 1 μM (1000 nM), the compounds have little affinity for the receptor tested.

The selectivity of the compounds according to the invention for the V₂ receptor can be demonstrated by the ratios calculated between the different values of IC₅₀ measured on each receptor and the IC₅₀ measured on the V₂ receptor: the higher this ratio, the higher the selectivity of the compounds according to the invention for the V₂ receptors. In this instance, as shown in Table A, the IC₅₀ ratios of the compounds according to the invention are much higher than 10, thus demonstrating their selectivity.

The compounds according to the invention, having affinity and selectivity for the V₂ receptors, display good pharmacological properties and are particularly suitable for use in the preparation of medicinal products, especially of medicinal products that are antagonists of binding to the V₂ receptors.

According to another of its aspects, the invention therefore relates to medicinal products that comprise at least one compound of formula (I).

Compounds that are antagonists of the V₂ vasopressin receptors display aquaretic properties in animals and humans (Cardiovascular Drug Review, (2001), 3: pp. 201-214). Thus, the compounds according to the invention possess a broad range of therapeutic indications and can advantageously replace conventional diuretics in all the pathologies where they are recommended for humans and animals.

Thus, the compounds according to the invention may be useful notably in the treatment and/or prevention of disorders of the central and peripheral nervous systems, of the cardiovascular system, of the endocrine and hepatic system, of the renal system, of the gastric, intestinal and pulmonary system, in opthalmology and in problems of sexual behaviour, in humans and in animals.

More particularly, the compounds according to the invention can be used in the treatment and/or prevention of various vasopressin-dependent disorders as well as in dysfunction of vasopressin secretion such as the inappropriate syndrome of secretion of vasopressin (or “SIADH”, for Syndrome of Inappropriate ADH Secretion), cardiovascular disorders, such as hypertension, pulmonary hypertension, heart failure, circulatory failure, myocardial infarction, atherosclerosis or coronary vasospasm, especially in smokers, unstable angina and percutaneous transluminal coronary angioplasty (PTCA), ischaemic heart disease, disturbances of haemostasis notably haemophilia, Von Willebrand syndrome; central nervous system disorders, pain, migraine, cerebral vasospasm, cerebral haemorrhage, cerebral oedema, depression, anxiety, bulimia, psychotic, states, for example memory problems; renopathies and renal dysfunction such as oedema, renal vasospasm, necrosis of the renal cortex, nephrotic syndrome, polycystic kidney diseases (PKD) in their various forms in children and in adults, hyponatraemia and hypokalaemia, diabetes, diabetic nephropathies, nephrogenic diabetes insipidus (NDI), NSIADH (nephrogenic syndrome of inappropriate antidiuresis), Schwartz-Bartter syndrome or renal lithiasis, urinary tract infections; disorders of the gastric system, such as gastric vasospasm, portal hypertension, hepatocirrhosis, ulcers, vomiting pathology, for example nausea including nausea due to chemotherapy, motion sickness, diabetes insipidus and enuresis; disorders of the hepatic system such as liver cirrhoses; abdominal ascites and all disorders causing abnormal water retention; adrenal disorders (Cushing disease) and in particular hypercorticism and hyperaldosteronaemia. The compounds according to the invention can also be used in the treatment and/or prevention of problems of sexual behaviour, in overweight conditions or excess weight and obesity, advantageously replacing the conventional diuretics already used for this indication. In women, compounds according to the invention can be used for treating dysmenorrhoea or premature labour. The compounds according to the invention can also be used in the treatment of small cell lung cancers, hyponatraemic encephalopathies, Raynaud disease, pulmonary syndrome, glaucoma and prevention of cataract, in postoperative treatments, notably after abdominal, cardiac or haemorrhagic surgery and in treatments of disorders or diseases of the inner ear such as Ménière disease, tinnitus, vertigo, hearing difficulties, notably for low tones, or buzzing, hydrops and notably endolymphatic hydrops, osteoporosis.

According to another of its aspects, the present invention relates to pharmaceutical compositions comprising, as active principle, at least one compound according to the invention. These pharmaceutical compositions contain an effective dose of at least one compound of formula (I) according to the invention, as well as at least one pharmaceutically acceptable excipient.

Said excipients are selected according to the dosage form and the desired method of administration, from the usual excipients that are known by a person skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or any salt, solvate or hydrate thereof, can be administered in a unit dosage form, mixed with conventional pharmaceutical excipients, to animals and to human beings for the prophylaxis or treatment of the aforementioned disorders or diseases.

The appropriate unit dosage forms comprise the oral forms such as tablets, soft or hard capsules, powders, granules and oral solutions or suspensions, forms for sublingual, buccal, intratracheal, intraocular, intranasal administration or administration by inhalation, forms for topical, transdermal, subcutaneous, intramuscular or intravenous administration, forms for rectal administration and implants. For topical application, the compounds according to the invention can be used in creams, gels, ointments or lotions. As an example, a unit dosage form of a compound according to the invention in the form of a tablet can comprise the following components:

| | |
|---|---|
| Compound according to the example of the invention | 50.0 mg |
| Mannitol | 223.75 mg |
| Croscarmellose sodium | 6.0 mg |
| Maize starch | 15.0 mg |
| Hydroxypropyl methylcellulose | 2.25 mg |
| Magnesium stearate | 3.0 mg |

Said unit forms are dosed to provide a daily administration from 0.5 mg to 800 mg of active principle per individual, more particularly from 0.5 mg to 200 mg, depending on the galenical form.

There may be cases when higher or lower dosages are appropriate; said dosages are still within the scope of the invention. According to the usual practice, the appropriate dosage for each patient is determined by the doctor according to the method of administration, and said patient's weight and response.

The present invention, according to another of its aspects, also relates to a method of treatment and/or prevention of the aforementioned pathologies, which comprises the administration, to a patient, of an effective dose of a compound according to the invention, or of one of its hydrates or solvates.

We claim:

1. A compound of formula (I):
wherein 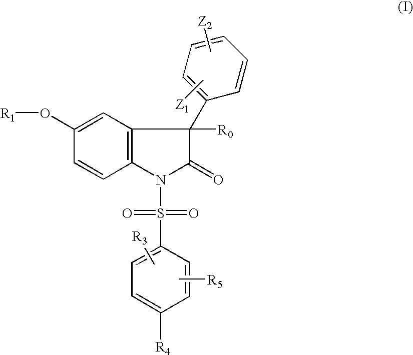
R₀ is (C₁-C₄)alkyl, mono or polyfluoro-(C₁-C₄)alkyl, —(CH₂)ₙ-cyclopropyl, (C₂-C₄)alkenyl or (C₂-C₄)alkynyl;
R₁ is hydrogen, (C₁-C₅)alkyl, mono or polyfluoro-(C₁-C₅)alkyl, hydroxy-(C₁-C₅)alkyl or —(CH₂)ₘ—(C₃-C₅)cycloalkyl;
Z1 is hydrogen, halogen, (C₁-C₄)alkyl, mono or polyfluoro-(C₁-C₄)alkyl, (C₁-C₄)alkoxy, mono or polyfluoro-(C₁-C₄)alkoxy, or —(CH₂)ₙ-cyclopropyl, wherein said cyclopropyl group is optionally substituted with one or more fluorine atoms;
Z₂ is halogen or T₁W,
wherein
T₁ is —(CH₂)ₙ—;
W is hydrogen, (C₁-C₄)alkyl, mono or polyfluoro-(C₁-C₄)alkyl or cyclopropyl, wherein said cyclopropyl group is optionally substituted with one or more fluorine atoms; —C(O)NR₆R₇
wherein R₆ and R₇ are, independently of one another, hydrogen, (C₁-C₆)alkyl, mono or polyfluoro-(C₁-C₆)alkyl, —(CH₂)ₘ—(C₃-C₆)cycloalkyl, wherein said cycloalkyl group is optionally substituted with one or more fluorine atoms, hydroxyl or NRR′; or
R₆ and R₇ are, independently of one another, —(CH₂)ₚ-pyrrolidinyl, —(CH₂)ₚ-piperidyl, —(CH₂)ₚ-pyridyl,
wherein said pyrrolidinyl, piperidyl and pyridyl groups are optionally substituted with one or more halogen atoms, (C₁-C₄)alkyl, mono or polyfluoro(C₁-C₄)alkyl, benzyl or —OR,
—(CH₂)_{q}—NRₐR_{b},
wherein Rₐ and R_{b} are, independently of one another, hydrogen, (C₁-C₄)alkyl, mono or polyfluoro-(C₁-C₄)alkyl, —(CH₂)ₐ-cyclopropyl, wherein said cyclopropyl is optionally substituted with one or more fluorine atoms; or
Rₐ and R_{b} form, together with the nitrogen atom to which they are attached, a monocyclic heterocyclic group, said monocyclic heterocyclic group being optionally substituted with one or more hydroxyl, (C₁-C₄)alkyl, mono or polyfluoro-(C₁-C₄)alkyl, (C₁-C₄)alkoxy, mono or polyfluoro(C₁-C₄)alkoxy groups, or by —NRR′;
wherein R′ and R are, independently of one another, hydrogen, (C₁-C₄)alkyl or mono or polyfluoro-(C₁-C₄)alkyl;
—(CH₂)ₛ—C(O)NRₐR_{b},
—(CH₂)_{q}—OR; or
R₆ and R₇ form, together with the nitrogen atom to which they are attached, a monocyclic heterocycle optionally substituted with one or more fluorine atoms, one or more (C₁-C₄)alkyl, mono or polyfluoro-(C₁-C₄)alkyl, —NR′R, or —OR groups; or
R₆ and R₇ form, together with the nitrogen atom to which they are attached, a bicyclic heterocycle optionally substituted with one or more fluorine atoms, (C₁-C₄)alkyl, mono or polyfluoro-(C₁-C₄)alkyl, —OR or —NR′R groups;
—NR₈C(O)R₉,
wherein
R₈ is hydrogen, (C₁-C₄)alkyl or mono or polyfluoro-(C₁-C₄)alkyl,
R₉ is hydrogen, (C₁-C₄)alkyl group, mono or polyfluoro-(C₁-C₄)alkyl, —(CH₂)ᵣ—NR_{d}Rₑ,
wherein R_{d} and Rₑ are, independently of one another, hydrogen, halogen, C₁-C₆)alkyl or mono or polyhalogeno-(C₁-C₆)alkyl, or
R_{d} and Rₑ form, together with the nitrogen atom to which they are attached, a monocyclic heterocyclic group optionally substituted with one or more fluorine atoms, one or more (C₁-C₄)alkyl groups or mono or polyfluoro-(C₁-C₄)alkyl groups or —OR;
—(CH₂)ₘ-pyrrolidinyl, —(CH₂)ₘ-piperidyl or —(CH₂)ₘ-pyridyl, wherein said pyrrolidinyl, piperidyl, and pyridyl groups are optionally substituted with one or more (C₁-C₄)alkyl, halogen, mono or polyfluoro-(C₁-C₄)alkyl or benzyl groups;
—NR₁₀R₁₁
wherein
R₁₀ and R₁₁ are, independently of one another, hydrogen, hydroxyl, (C₁-C₆)alkyl or a mono or polyfluoro-(C₁-C₆)alkyl group; or
R₁₀ and R₁₁ form, together with the nitrogen atom to which they are attached, a monocyclic heterocycle optionally substituted with one or more (C₁-C₄)alkyl groups, a mono or polyfluoro-(C₁-C₄)alkyl group or oxo;
—OR₁₂
wherein
R₁₂ is hydrogen, (C₁-C₄)alkyl, mono or polyfluoro-(C₁-C₄)alkyl, benzyl or —(CH₂)_{q}—NR′R;
—C(O)OR₁₉
wherein R₁₉ is hydrogen, (C₁-C₆)alkyl, mono or polyfluoro-(C₁-C₆)alkyl, —(CH₂)_{q}—NRₐR_{b}, —(CH₂)_{q}—OR, —(CH₂)ₚ-pyrrolidinyl or —(CH₂)ₚ-piperidyl, wherein said pyrrolidinyl and piperidyl groups are optionally substituted with one or more fluorine atoms, (C₁-C₄)alkyl or mono or polyfluoro-(C₁-C₄)alkyl;
R₄ is (C₁-C₄)alkyl, mono or polyfluoro-(C₁-C₄)alkyl, —OR, (C₂-C₄)alkenyl, nitro, COOR_{c},
wherein R_{c} is hydrogen, (C₁-C₄)alkyl, mono or polyfluoro-(C₁-C₄)alkyl or benzyl;
benzyloxy,
—C(O)NR₁₃R₁₄;
wherein R₁₃ and R₁₄ are, independently of one another, hydrogen, (C₁-C₆)alkyl, said alkyl group being optionally substituted with one or more fluorine atoms, hydroxyl, —NRR′, (C₁-C₆)alkyloxycarbonylamino or (C₁-C₆)alkyloxycarbonyl, or
R₁₃ and R₁₄ are, independently of one another, —(CH₂)ₙ—(C₃-C₆)cycloalkyl, wherein said cycloalkyl group is optionally substituted with one or more fluorine atoms,
—NR₁₅R₁₆;
wherein R₁₅ and R₁₆ are, independently of one another, hydrogen, hydroxyl, (C₁-C₄)alkyl, mono or polyfluoro-(C₁-C₄)alkyl or —(CH₂)ₙ—(C₃-C₅)cycloalkyl optionally substituted with one or more fluorine atoms;
or R₁₅ and R₁₆ form, together with the nitrogen atom to which they are attached, a monocyclic heterocycle; or
—NR₁₇C(O)R₁ₛ;
wherein
R₁₇ is hydrogen, (C₁-C₄)alkyl or mono or polyfluoro-(C₁-C₄)alkyl,
R₁₈ is (C₁-C₆)alkyl, mono or polyfluoro-(C₁-C₆)alkyl, —(CH₂)ₙ—(C₃-C₆)cycloalkyl, —NR_{d}Rₑ, phenyl wherein said phenyl group is optionally substituted with one or more (C₁-C₄)alkyl groups or a mono or polyfluoro-(C₁-C₄)alkyl group;
R₃ and R₅ are, independently of one another, hydrogen, halogen, (C₁-C₄)alkyl, mono or polyfluoro-(C₁-C₄)alkyl, (C₁-C₄)alkoxy or mono or polyfluoro-(C₁-C₄)alkoxy;
m is 0, 1 or 2;
n is 0 or 1;
p is 0, 1, 2 or 3;
q is 2, 3, 4 or 5;
r is 0, 1, 2, 3 or 4; and
s is 1, 2 or 3;
or hydrate or solvate or pharmaceutically acceptable salt thereof, in the form of cis/trans isomers or mixtures thereof.

2. The compound of claim 1 wherein R₀ is (C₁-C₃)alkyl, or a hydrate or solvate or pharmaceutically acceptable salt thereof, in the form of cis/trans isomers or mixtures thereof.

3. The compound of claim 2 wherein R₁ is (C₁-C₅)alkyl, or a hydrate or solvate or pharmaceutically acceptable salt thereof, in the form of cis/trans isomers or mixtures thereof.

4. The compound of claim 3 wherein R₀ is methyl and R₁ is (C₁-C₂)alkyl, or a hydrate or solvate or pharmaceutically acceptable salt thereof, in the form of cis/trans isomers or mixtures thereof.

5. The compound of claim 4 wherein Z₁ is halogen, or a hydrate or solvate or pharmaceutically acceptable salt thereof, in the form of cis/trans isomers or mixtures thereof.

6. The compound of claim 5 wherein Z₂ is T₁W, wherein T₁ is —(CH₂)ₙ— with n equal to 0 and W is —C(O)OR₁₉ or —C(O)NR₆R₇, wherein R₁₉, R₆ and R₇ are as defined in claim 1, or a hydrate or solvate or pharmaceutically acceptable salt thereof, in the form of cis/trans isomers or mixtures thereof.

7. The compound of claim 6 wherein Z₁ is in position −2; or a hydrate or solvate or pharmaceutically acceptable salt thereof, in the form of cis/trans isomers or mixtures thereof.

8. The compound of claim 7 wherein Z₂ is in position −5; or a hydrate or solvate or pharmaceutically acceptable salt thereof, in the form of cis/trans isomers or mixtures thereof.

9. The compound of claim 8 wherein R₄ is —C(O)NR₁₃R₁₄, wherein R₁₃ and R₁₄ are as defined in claim 1, or a hydrate or solvate or pharmaceutically acceptable salt thereof, in the form of cis/trans isomers or mixtures thereof.

10. The compound of claim 5 wherein Z₂ is T₁W, wherein T₁ is —(CH₂)ₙ— with n equal to 0 and W is −OR₁₂ wherein R₁₂ is hydrogen, or a hydrate or solvate or pharmaceutically
acceptable salt thereof, in the form of cis/trans isomers or mixtures thereof.

11. The compound of claim 5 wherein Z₁ is in position −2 and Z₂ is halogen, or a hydrate or solvate or pharmaceutically acceptable salt thereof, in the form of cis/trans isomers or mixtures thereof.

12. The compound of claim 1 which is
N-tert-butyl-4-[3-(2-chlorophenyl)-5-ethoxy-3-methyl-2-oxo-2,3-dihydroindole-1-sulphonyl]benzamide (Example 2);
N-tert-butyl-4-[3-(2-fluorophenyl)-5-ethoxy-3-methyl-2-oxo-2,3-dihydroindole-1-sulphonyl]benzamide (Example 6);
methyl 3-[1-(4-tert-butylcarbamoylbenzenesulphonyl)-5-ethoxy-3-methyl-2-oxo-2,3-dihydro-1H-indol-3-yl]-4-chlorobenzoate (Example 9);
N-tert-butyl-4-[3-(2-chlorophenyl)-5-ethoxy-3-methyl-2-oxo-2,3-dihydroindole-1-sulphonyl]benzamide (Example 12);
N-cyclopentyl-4-[3-(2-chlorophenyl)-5-ethoxy-3-methyl-2-oxo-2,3-dihydroindole-1-sulphonyl]benzamide (Example 13);
N-tert-butyl-3-methoxy-4-[3-(2-chlorophenyl)-5-ethoxy-3-methyl-2-oxo-2,3-di-hydroindole-1-sulphonyl]benzamide (Example 15);
N-tert-butyl-3-methoxy-4-[3-(2-fluorophenyl)-5-ethoxy-3-methyl-2-oxo-2,3-dihydro-indole-1-sulphonyl]benzamide (Example 16);
3-[1-(4-tert-butylcarbamoyl-2-methoxybenzenesulphonyl)-5-ethoxy-3-methyl-2-oxo-2,3-dihydro-1H-indol-3-yl]-4-chlorobenzoic acid (Example 19);
4-chloro-3-[1-(4-tert-butylcarbamoyl-2-methoxybenzenesulphonyl)-5-ethoxy-3-methyl-2-oxo-2,3-dihydro-1H-indol-3-yl]-N-ethylbenzamide (Example 21);
4-chloro-3-[1-(4-tert-butylcarbamoyl-2-methoxybenzenesulphonyl)-5-ethoxy-3-methyl-2-oxo-2,3-dihydro-1H-indol-3-yl]-N-(2-pyridyl)benzamide (Example 22);
4-chloro-3-[1-(4-tert-butylcarbamoyl-2-methoxybenzenesulphonyl)-5-ethoxy-3-methyl-2-oxo-2,3-dihydro-1H-indol-3-yl]-N-(3-dimethylaminopropyl)benzamide (Example 25);
4-chloro-3-[1-(4-tert-butylcarbamoyl-2-methoxybenzenesulphonyl)-5-ethoxy-3-methyl-2-oxo-2,3-dihydro-1H-indol-3-yl]-N-(1-methylpiperidin-4-yl)benzamide (Example 28);
4-chloro-3-[1-(4-tert-butylcarbamoyl-2-methoxybenzenesulphonyl)-5-ethoxy-3-methyl-2-oxo-2,3-dihydro-1H-indol-3-yl]-N-(2-pyrrolidin-1-ylethyl)benzamide (Example 29);
4-chloro-3-[1-(4-tert-butylcarbamoyl-2-methoxybenzenesulphonyl)-5-ethoxy-3-methyl-2-oxo-2,3-dihydro-1H-indol-3-yl]-N-(2-diethylaminoethyl)benzamide (Example 33);
4-chloro-3-[1-(4-tert-butylcarbamoyl-2-methoxybenzenesulphonyl)-5-ethoxy-3-methyl-2-oxo-2,3-dihydro-1H-indol-3-yl]-N-(2-morpholin-4-ylethyl)benzamide (Example 36);
4-chloro-3-[1-(4-tert-butylcarbamoyl-2-methoxybenzenesulphonyl)-5-ethoxy-3-methyl-2-oxo-2,3-dihydro-1H-indol-3-yl]-N[2-(4-methylpiperazin-1-yl)ethyl]benzamide (Example 37);
4-chloro-3-[1-(4-tert-butylcarbamoyl-2-methoxybenzenesulphonyl)-5-ethoxy-3-methyl-2-oxo-2,3-dihydro-1H-indol-3-yl]-N-[3-(4-methylpiperazin-1-yl)propyl]benzamide (Example 40);
4-chloro-3-[1-(4-tert-butylcarbamoyl-2-methoxybenzenesulphonyl)-5-ethoxy-3-methyl-2-oxo-2,3-dihydro-1H-indol-3-yl]-N—(R)-pyrrolidin-3-ylbenzamide (Example 43);
4-chloro-3-[1-(4-tert-butylcarbamoyl-2-methoxybenzenesulphonyl)-5-ethoxy-3-methyl-2-oxo-2,3-dihydro-1H-indol-3-yl]-N-piperidin-4-ylbenzamide (Example 44);
4-chloro-3-[1-(4-tert-butylcarbamoyl-2-methoxybenzenesulphonyl)-5-ethoxy-3-methyl-2-oxo-2,3-dihydro-1H-indol-3-yl]-N-2-aminoethylbenzamide (Example 47);
3-[1-(4-tert-butylcarbamoylbenzenesulphonyl)-5-ethoxy-3-methyl-2-oxo-2,3-di-hydro-1H-indol-3-yl]-4-chlorobenzoic acid (Example 48);
4-chloro-3-[1-(4-tert-butylcarbamoylbenzenesulphonyl)-5-ethoxy-3-methyl-2-oxo-2,3-dihydro-1H-indol-3-yl]-N-(1-methylpiperidin-4-yl)benzamide (Example 51);
4-chloro-3-[1-(4-tert-butylcarbamoylbenzenesulphonyl)-5-ethoxy-3-methyl-2-oxo-2,3-dihydro-1H-indol-3-yl]-N-(2-diethylaminoethyl)benzamide (Example 53);
N-tert-butyl-4-{3-[2-chloro-5-(3-dimethylaminopropionylamino)phenyl]-5-ethoxy-3-methyl-2-oxo-2,3-dihydroindole-1-sulphonyl}-3-methoxybenzamide (Example 58);
1-methylpiperidine-4-carboxylic acid {3-[1-(4-tert-butylcarbamoyl-2-methoxy-benzenesulphonyl)-5-ethoxy-3-methyl-2-oxo-2,3-dihydro-1H-indol-3-yl]-4-chlorophenyl}-amide (Example 62);
N-(2-fluoro-1,1-dimethylethyl)-4-[3-(2-chlorophenyl)-5-ethoxy-3-methyl-2-oxo-2,3-dihydroindole-1-sulphonyl]benzamide (Example 66);
4-[3-(2-chlorophenyl)-5-ethoxy-3-methyl-2-oxo-2,3-dihydroindole-1-sulphonyl]-N-(2-hydroxy-1,1-dimethylethyl)benzamide (Example 72);
4-chloro-3-[1-(4-tert-butylcarbamoylbenzenesulphonyl)-5-ethoxy-3-methyl-2-oxo-2,3-dihydro-1H-indol-3-yl]-N[2-(4-hydroxypiperidin-1-yl)ethyl]benzamide (Example 79);
4-chloro-3-[1-(4-tert-butylcarbamoylbenzenesulphonyl)-5-ethoxy-3-methyl-2-oxo-2,3-dihydro-1H-indol-3-yl]-N-2,2,2-trifluoroethylbenzamide (Example 80);
4-chloro-3-[1-(4-tert-butylcarbamoylbenzenesulphonyl)-5-ethoxy-3-methyl-2-oxo-2,3-dihydro-1H-indol-3-yl]-N-2-hydroxyethylbenzamide (Example 83);
4-chloro-3-[1-(4-tert-butylcarbamoylbenzenesulphonyl)-5-ethoxy-3-methyl-2-oxo-2,3-dihydro-1H-indol-3-yl]-N-cyclopentylbenzamide (Example 87);
4-chloro-3-[1-(4-tert-butylcarbamoylbenzenesulphonyl)-5-ethoxy-3-methyl-2-oxo-2,3-dihydro-1H-indol-3-yl]-N-[trans-(4-hydroxycyclohexyl)]benzamide (Example 88);
4-chloro-3-[1-(4-tert-butylcarbamoylbenzenesulphonyl)-5-ethoxy-3-methyl-2-oxo-2,3-dihydro-1H-indol-3-yl]-N-[trans-(4-dimethylaminocyclohexyl)]benzamide (Example 90);
N-tert-butyl-4-[3-methyl-3-phenyl-5-ethoxy-2-oxo-2,3-dihydroindole-1-sulphonyl]-benzamide (Example 98);
N-tert-butyl-3-methoxy-4-[3-methyl-3-phenyl-5-ethoxy-2-oxo-2,3-dihydroindole-1-sulphonyl]benzamide (Example 99);
N-tert-butyl-4-[3-methyl-3-(2-fluorophenyl)-5-ethoxy-2-oxo-2,3-dihydroindole-1-sulphonyl]benzamide (Example 101); or
N-tert-butyl-4-[3-methyl-3-(2-chloro-6-fluorophenyl)-5-ethoxy-2-oxo-2,3-dihydro-indole-1-sulphonyl]benzamide (Example 125);
or a hydrate or solvate or pharmaceutically acceptable salt thereof, in the form of cis/trans isomers or mixtures thereof.

13. A method of preparing a compound of formula (I) of claim 1 comprising: reacting a compound of formula (II): 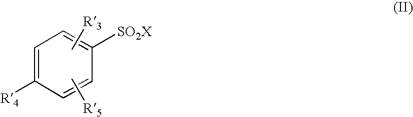
wherein R′₃, R′₄ and R′₅ are respectively, and independently of one another, precursor groups of the groups R₃, R₄ and R₅ or, alternatively, represent the groups R₃, R₄ and R₅ as defined for the compounds of formula (I), and X represents a halogen atom,
with a compound of formula (III): 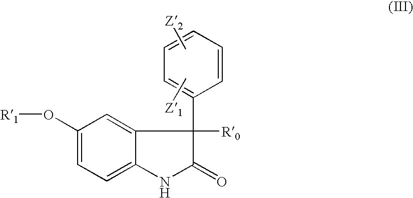
wherein R′₀, R′₁, Z′₁ and Z′₂ are respectively, and independently of one another, precursor groups of the groups R₀, R₁, Z₁ and Z₂, or alternatively the groups R₀, R₁, Z₁ and Z₂ as defined for the compounds of formula (I),
in the presence of a metal hydride, at temperatures between −40° and 25° C., in an anhydrous solvent,
or alternatively, the compounds of formula (I) are obtained indirectly, via the compounds of formula (I′): 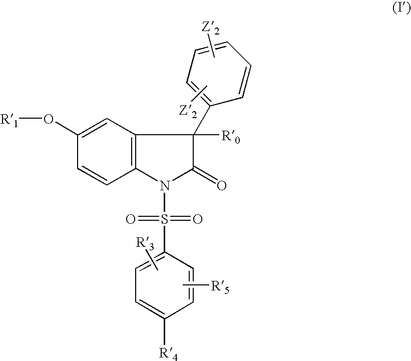
wherein R′₀, R′₁, Z′1, Z′₂, R′₃, R′₄ and R′₅ represent respectively, and independently of one another, precursor groups of the groups R₀, R₁, Z₁, Z₂, R₃, R₄ and R₅, or alternatively the groups R₀, R₁, Z₁, Z₂, R₃, R₄ and R₅ as defined for the compounds of formula (I), themselves obtained by reaction of a compound of formula (II) with a compound of formula (III) as defined previously.

14. A pharmaceutical composition comprising a compound of claim 1 and a pharmaceutically acceptable carrier.

15. A method of treating one or more disorders selected from the group consisting of:
disorders of the central or peripheral nervous systems;
disorders of the cardiovascular system;
disorders of the endocrine system;
disorders of the hepatic system;
disorders of the renal system;
disorders of the gastric system;
disorders of the intestinal system;
disorders of the pulmonary system;
opthalmologic disorders; and
sexual behaviour disorders,
comprising administering to a patient in need thereof a therapeutically effective amount of a compound of claim 1.

16. A method of treating one or more disorders or conditions selected from the following: vasopressin-dependent disorders as well as dysfunctions of vasopressin secretion such as the inappropriate syndrome of vasopressin secretion, cardiovascular disorders, such as hypertension, pulmonary hypertension, heart failure, circulatory failure, myocardial infarction, atherosclerosis or coronary vasospasm, in particular in smokers, unstable angina and percutaneous transluminal coronary angioplasty, ischaemic heart disease, disturbances of haemostasis notably haemophilia, Von Willebrand syndrome; disorders of the central nervous system, pain, migraine, cerebral vasospasm, cerebral haemorrhage, cerebral oedema, depression, anxiety, bulimia, psychotic states, for example memory disorders; renopathies and renal dysfunction such as oedema, renal vasospasm, necrosis of the renal cortex, nephrotic syndrome, renal polycystic diseases in their various forms in children and in adults, hyponatraemia, hypokalaemia, diabetes, diabetic nephropathies, nephrogenic diabetes insipidus, “NSIADH”, Schwartz-Bartter syndrome or renal lithiasis, urinary tract infections; disorders of the gastric system, such as gastric vasospasm, portal hypertension, hepatocirrhosis, ulcers, vomiting pathology, for example nausea including nausea due to chemotherapy, motion sickness, diabetes insipidus and enuresis; disorders of the hepatic system such as hepatic cirrhosis; abdominal ascites and all disorders causing abnormal water retention; adrenal disorders, Cushing disease, hypercorticism and hyperaldosteronaemia, problems of sexual behaviour, overweight conditions or excessive weight and obesity, in dysmenorrhoea or premature labour, small cell lung cancers, hyponatraemic encephalopathies, Raynaud disease, pulmonary syndrome, glaucoma and prevention of cataract, in postoperative treatments, notably after abdominal, cardiac or haemorrhagic surgery and in treatments of disorders or diseases of the inner ear such as Ménière disease, tinnitus, vertigo, hearing difficulties, notably at low tones, or buzzing, hydrops and notably endolymphatic hydrops, osteoporosis,
comprising administering to a patient in need thereof a therapeutically effective amount of a compound of claim 1.
